(19) United States
(12) Patent Application Publication
(10) Pub. No.: US 2010/0305186 A1
(43) Pub. Date: Dec. 2, 2010

(54) METHODS FOR MEDIATING GENE SUPPRESSION

(75) Inventors: Gregory Martin Arndt, New South Wales (AU); Mitch Raponi, New South Wales (AU)

Correspondence Address: Sullivan & Worcester LLP, 1666 K Street, N.W., Washington, DC, 20006, US

(73) Assignee: JOHNSON & JOHNSON RESEARCH PTY LIMITED, Eveleigh (AU)

(21) Appl. No.: 10/297,167

(22) PCT Filed: May 29, 2001
(86) PCT No.: PCT/AU2001/000627

§ 371 Date: Jul. 5, 2006

(30) Foreign Application Priority Data
May 30, 2000 (AU) PQ7830
Aug. 7, 2000 (AU) PQ9246

(51) Int. Cl. A61K 31/7105; C12N 1/14; A61K 31/7088; C12N 1/15; A61P 43/00

(52) U.S. Cl. 514/44 A; 435/254.1; 514/44 R; 435/254.11

(57) ABSTRACT

The present invention is concerned with methods for enhancing gene suppression in cells and in particular it is concerned with improved methods for enhancing RNAi-mediated gene silencing by manipulation of factors associated with RNAi. The present invention is also concerned with methods for identifying factors which down-regulate as well as those which up-regulate RNAi. It is also concerned with genetic constructs useful for enhancing or modulating gene silencing and cells harbouring such constructs.

### TECHNICAL FIELD

The present invention is concerned with methods for enhancing gene suppression in cells and in particular it is concerned with improved methods for enhancing RNAi-mediated gene silencing by manipulation of factors associated with RNAi. The present invention is also concerned with methods for identifying factors which down-regulate as well as those which up-regulate RNAi. It is also concerned with genetic constructs useful for enhancing or modulating gene silencing and cells harbouring such constructs.

### BACKGROUND OF THE INVENTION

The use of double-stranded RNA (dsRNA) to specifically interfere with gene expression has received considerable attention because of its demonstrated potency in a range of organisms, including some which have so far been genetically intractable. Termed RNA interference (RNAi), it has been implicated in viral defense, control of transpositional elements, genetic imprinting and endogenous gene regulation. It has been hypothesised to be the central mechanism in post-transcriptional gene silencing (PTGS), co-suppression, quelling, and antisense RNA-mediated gene suppression. One model that has been proposed is that dsRNA is fragmented into 21-25 nt species by dsRNA-specific nucleases, amplified by RNA-dependent RNA polymerase, and then dissociated and free to attack homologous mRNA by RNA nuclease-mediated degradation. The application of this technique will greatly facilitate the dissection of gene function and the validation of genes involved in disease states.

Recently at least two different strategies have been undertaken to identify the cellular proteins composing a proposed multi-protein complex involved in the recognition of dsRNA and the activation of dsRNA-mediated gene interference. The first involves the use of classical chemical mutagenesis or insertional mutagenesis to isolate mutants completely defective for RNAi and cloning of the relevant genes using complementation. These studies have been carried out in genetically tractable organisms such as plants, worms and fungi. The genetic screens described involve the use of RNAi systems in which the degree of suppression is complete. The mutagenesis produces mutants in which the RNAi effect is completely reversed indicating the loss of a cellular factor (function) required for the RNAi effect. Thus these genetic screens would most likely miss factors that have subtle effects or rate limiting or rate determining roles in RNAi.

The second strategy for finding key players in RNAi has involved the use of cell free assays. These in vitro reconstitution assays, on the other hand, identify cellular factors that impact on RNAi outside of the cellular context and therefore the cellular role of these factors must always be tested.

However, the major disadvantages of these strategies are that genes will not be identified if they are essential to the organism, nor will they directly identify gene activities which will enhance RNAi when overexpressed.

Thus there is a need for models which can demonstrate a range of RNAi efficacies, with both increasing and decreasing quantitative activities being selectable. This would enable the identification of factors which can enhance or reduce the gene silencing effect.

It is therefore an object of the present invention to overcome or at least ameliorate one or more disadvantages of the prior art, or provide a useful alternative.

### SUMMARY OF THE INVENTION

Through the use of a fission yeast model for the study of dsRNA-mediated gene silencing and in the search for factors involved in this process, it was surprisingly found that the natural level of RNAi activity can be enhanced by manipulating factors associated with RNAi activity or efficacy. Thus, it has been found that by increasing the steady-state levels of a target nucleic acid sequence in the presence of the same pool of the corresponding antisense sequence, or a part thereof, the antisense-mediated suppression was not only maintained, but enhanced. This is indicative of an RNAi-like mechanism of gene suppression. It has also been found that overexpression of certain sequences, named herein RNAi enhancing sequences (res), (also referred to herein as anti-sense enhancing sequences—aes), also had the ability to enhance RNAi.

This ability of RNAi activity to be enhanced in S. pombe provides a model system which enables the analysis of RNAi processes and the identification and study of factors which either up-regulate or down-regulate its activity. The system has been further used to identify RNAi enhancing gene sequences which increase PTGS efficacy when their resulting protein activities are augmented in vivo. The model used to exemplify the present invention and the methods described are also applicable to treatment of disorders in which gene expression requires more efficient modulation or silencing.

According to a first aspect there is provided a method for inhibiting the expression of a target nucleic acid in a cell, which method comprises the steps of

(i) elevating in the cell the level of an RNAi factor, and
(ii) prior, concurrently with or subsequent to performing step (i), introducing into the cell a molecule which is, or gives rise to, an anti-sense nucleic acid directed toward at least a portion of the RNA transcript of the target nucleic acid, under conditions permitting the RNAi factor to increase the degree to which the anti-sense nucleic acid inhibits expression of the target nucleic acid.

According to a second aspect there is provided a method of increasing cellular susceptibility to anti-sense-mediated inhibition of target nucleic acid expression, which method comprises elevating the level of an RNAi factor in a cell that expresses said target nucleic acid, with the proviso that the cell is to have prior, concurrently or subsequently introduced thereinto a molecule which is, or gives rise to, an anti-sense nucleic acid directed toward at least a portion of the RNA transcript of the target nucleic acid under conditions permitting the RNAi factor to increase the degree to which the anti-sense nucleic acid inhibits expression of the target nucleic acid.

According to a third aspect there is provided a method for treating a subject suffering from a disorder whose alleviation is mediated by inhibiting the expression of a target nucleic acid, which method comprises the steps of

(i) elevating the level of an RNAi factor in the subject's cells where the target nucleic acid is expressed, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting the RNAi factor to increase the degree to which the anti-sense nucleic acid inhibits expression of the target nucleic acid, thereby treating the subject.

According to a fourth aspect there is provided a method for inhibiting in a subject the onset of a disorder whose alleviation is mediated by inhibiting the expression of a target nucleic acid, which method comprises the steps of

(i) elevating the level of an RNAi factor in the subject's cells where the target nucleic acid would be expressed if the subject were suffering from the disorder, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting the RNAi factor to increase the degree to which the anti-sense nucleic acid would inhibit expression of the target nucleic acid were such expression to occur, thereby inhibiting in the subject the onset of the disorder.

According to a fifth aspect there is provided a method of determining whether inhibiting the expression of a particular target nucleic acid or the activity of its product may alleviate a disorder, which method comprises the steps of

(i) elevating the level of an RNAi factor in a cell whose phenotype correlates with that of a cell from a subject having the disorder;
(ii) prior, concurrently with or subsequent to performing step (i), introducing into the cell a molecule which is, or gives rise to, an anti-sense nucleic acid directed toward at least a portion of the RNA transcript of the target nucleic acid under conditions permitting the RNAi factor to increase the degree to which the anti-sense nucleic acid inhibits expression of the target nucleic acid; and
(iii) determining whether the cell's phenotype now correlates with that of a cell from a subject in whom the disorder has been alleviated or the disorder is not evident, thereby determining whether inhibiting the expression of the target nucleic acid or the activity of its product may alleviate the disorder.

In a preferred embodiment the target nucleic acid is an endogenous nucleic acid or a part thereof, but it may also be an exogenous sequence or part thereof.

Preferably the level of the RNAi factor is elevated by introducing into the cell additional copies of, or agents which give rise to, the RNAi factor. It will be understood therefore that up-regulating the expression of an endogenous RNAi factor will also achieve the same result and is contemplated herein as part of the invention.

Preferably the factor is selected from the group consisting of a gene, cDNA, RNA or a protein. More preferred is a factor selected from the group consisting of a transcriptional activator of the antisense nucleic acid, a component of the RNAi machinery, a component of the DNA replication machinery and a component of translational machinery. Even more preferred is an RNAi factor which is an res sequence.

Also for preference the factor can be selected from the group consisting of ATP-dependent RNA helicase (ded1), transcriptional factor thi1, DNA replication protein sna41, ribosomal protein L7a, elongation factor EF-Tu and res1 as herein defined.

Further preferred factors are represented by the res sequences which are obtainable from transformed cells designated herein W18, W20, W21, W23, W27, W28, W30, W32 and W47.

Preferably the res sequence is represented by any one of Seq ID Nos 1 to 4.

The preferred cell is a eukaryotic cell and even more preferred is a mammalian cell. In certain embodiments of the invention described herein the preferred cell is a Schizosaccharomyces pombe cell.

Preferably the antisense nucleic acid corresponds to a part only of the target nucleic acid.

According to a sixth aspect there is provided a pharmaceutical composition for use in performing the method of any one of the previous aspects comprising

(i) an expressible nucleic acid encoding, or capable of increasing or decreasing the expression of, an RNAi factor;
(ii) a nucleic acid encoding a molecule which is, or gives rise to, an anti-sense nucleic acid directed toward at least a portion of the RNA transcript of the target nucleic acid; and
(iii) a pharmaceutically acceptable carrier,
wherein the nucleic acids of (i) and (ii) may be situated on the same or different molecules.

According to a seventh aspect there is provided a pharmaceutical composition for use in performing the method of any one of claims 2 to 17 comprising

(i) an nucleic acid which is the target nucleic acid or a part thereof, or an expressible nucleic acid encoding a factor capable of elevating the intracellular level of the target nucleic acid;
(ii) a nucleic acid encoding a molecule which is, or gives rise to, an anti-sense nucleic acid directed toward at least a portion of the RNA transcript of the target nucleic acid; and
(iii) a pharmaceutically acceptable carrier,
wherein the nucleic acids of (i) and (ii) may be situated on the same or different molecules.

According to an eighth aspect there is provided a cell having increased susceptibility to anti-sense-mediated inhibition of a target nucleic acid expression, which cell (i) expresses a target nucleic acid and (ii) comprises an elevated level of an RNAi factor, with the proviso that the cell is to have introduced thereinto a molecule which is, or gives rise to, an anti-sense nucleic acid directed toward at least a portion of the RNA transcript of the target nucleic acid under conditions permitting the RNAi factor to increase the degree to which the anti-sense nucleic acid inhibits expression of the target nucleic acid.

For preference the cell is a eukaryotic cell but more preferred is a mammalian cell. As indicated above, in certain embodiments of the invention described herein the preferred cell is a Schizosaccharomyces pombe cell.

According to a ninth aspect there is provided a method for inhibiting the expression of a target nucleic acid in a cell, which method comprises the steps of

(i) augmenting the level of the target nucleic acid or a part thereof in the cell, and
(ii) prior, concurrently with or subsequent to performing step (i), introducing into the cell a molecule which is, or gives rise to, an anti-sense nucleic acid directed toward at least a portion of the RNA transcript of said target nucleic acid, under conditions permitting an increase in the degree to which the anti-sense nucleic acid inhibits expression of said target nucleic acid.

According to a tenth aspect there is provided a method of increasing cellular susceptibility to anti-sense-mediated inhibition of a target nucleic acid expression, which method comprises augmenting the level of the target nucleic acid or a part thereof in a cell expressing the target nucleic acid, with the proviso that the cell is to have prior, concurrently or subsequently introduced thereinto a molecule which is, or gives rise to, an anti-sense nucleic acid directed toward at least a portion of the RNA transcript of the target nucleic acid under conditions permitting the increase in the degree to which the anti-sense nucleic acid inhibits expression of the target nucleic acid.

According to an eleventh aspect there is provided a method for treating a subject suffering from a disorder whose alleviation is mediated by inhibiting the expression of a target nucleic acid, which method comprises the steps of

(i) augmenting the level of said target nucleic acid or a part thereof in the subject's cells where the target nucleic acid is expressed, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting an increase in the degree to which the anti-sense nucleic acid inhibits expression of the target nucleic acid, thereby treating the subject.

According to a twelfth aspect there is provided a method for inhibiting in a subject the onset of a disorder whose alleviation is mediated by inhibiting the expression of a target nucleic acid, which method comprises the steps of

(i) augmenting the level of the target nucleic acid or a part thereof in the subject's cells where the target nucleic acid would be expressed if the subject were suffering from the disorder, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting an increase in the degree to which the anti-sense nucleic acid would inhibit expression of the target nucleic acid were such expression to occur, thereby inhibiting in the subject the onset of the disorder.

According to a thirteenth aspect there is provided a method of determining whether inhibiting the expression of a particular target nucleic acid or the activity of its product may alleviate a disorder, which method comprises the steps of

(i) augmenting the level of the target nucleic acid in a cell whose phenotype correlates with that of a cell from a subject having the disorder;
(ii) prior, concurrently with or subsequent to performing step (i), introducing into the cell a molecule which is, or gives rise to, an anti-sense nucleic acid directed toward at least a portion of the RNA transcript of the target nucleic acid under conditions permitting an increase in the degree to which the anti-sense nucleic acid inhibits expression of the target nucleic acid; and
(iii) determining whether the cell's phenotype now correlates with that of a cell from a subject in whom the disorder has been alleviated or the disorder is not evident, thereby determining whether inhibiting the expression of the target nucleic acid or the activity of its product may alleviate the disorder.

In a preferred embodiment the target nucleic acid is an endogenous nucleic acid or a part thereof, but it may also be an exogenous sequence or part thereof. Preferably the level of the target nucleic acid is augmented by introducing into the cell additional copies of, or agents which are capable of inducing intracellular over-expression of, the target nucleic acid. Over-expression can be achieved for an endogenous as well as an exogenous target nucleic acid. Preferably the nucleic acid used for augmenting content of the target nucleic acid is a fragment, derivative or analogue of the target nucleic acid. However it will be understood that the entire native sequence of the target nucleic acid may be employed.

Conveniently, the target nucleic acid may be coupled to a selectable marker.

The preferred cell is a eukaryotic cell and even more preferred is a mammalian cell. In certain embodiments of the invention described herein the preferred cell is a Schizosaccharomyces pombe cell.

Preferably the antisense nucleic acid corresponds to a part only of the target nucleic acid.

According to a fourteenth aspect there is provided a cell having increased susceptibility to anti-sense-mediated inhibition of a target nucleic acid expression, which cell (i) expresses said target nucleic acid and (ii) comprises an elevated level of said target nucleic acid, with the proviso that the cell is to have introduced thereinto a molecule which is, or gives rise to, an anti-sense nucleic acid directed toward at least a portion of the RNA transcript of the target nucleic acid under conditions permitting the RNAi factor to increase the degree to which the anti-sense nucleic acid inhibits expression of the target nucleic acid.

The preferred cell is a eukaryotic cell and even more preferred is a Schizosaccharomyces pombe cell.

According to a fifteenth aspect there is provided a method of identifying a cellular factor capable of effecting and/or modulating expression of a target nucleic acid in a cell having the target nucleic acid and a nucleic acid which is an antisense of the target nucleic acid or part thereof, which method comprises over-expressing said factor in the cell and wherein the expression of the target nucleic acid is capable of being enhanced or only partially suppressed.

According to a sixteenth aspect there is provided a factor identified by the method of the fifteenth aspect.

The preferred factors can be selected from the group consisting of a gene, cDNA, RNA or a protein. More preferred are factors selected from the group consisting of a transcriptional activator or the antisense nucleic acid, a component of the RNAi machinery, a component of the DNA replication machinery and a component of translational machinery. Even more preferred is a factor having an res sequence.

Preferred factors can also be selected from the group consisting of ATP-dependent RNA helicase (ded1), transcriptional factor thi1, DNA replication protein sna41, ribosomal protein L7a, elongation factor EF-Tu and res1 as herein defined.

According to a seventeenth aspect there is provided an RNAi factor which is an res sequence obtainable from transformed cells designated herein W18, W20, W21, W23, W27, W28, W30, W32 and W47.

According to an eighteenth aspect there is provided an RNAi factor which is an res sequence represented by Seq ID Nos 1 to 4.

According to a nineteenth aspect there is provided a Schizosaccharomyces pombe cell having a target nucleic acid or a part thereof and a antisense nucleic acid or a part thereof which corresponds to the target nucleic acid or a part thereof, wherein the expression of the target nucleic acid is capable of being enhanced or only partially suppressed.

The term “inhibiting expression of a target nucleic acid” as used in the context of the present invention is intended to encompass, but not be limited to, reduction or elimination of gene expression, whether or not the target nucleic acid is a gene, or a part thereof, introduced into the cell or it is an endogenous gene.

The term “RNAi factor” is intended to include in its scope any naturally occurring, modified or synthetic molecule capable of enhancing RNAi activity. This definition includes in its scope the factors referred to herein as RNAi enhancing sequences (res). The term res may be used herein interchangeably with the term aes (anti-sense enhancing sequences).

It will be understood by those skilled in the art that “elevating RNAi factor level” can be achieved by transfection, upregulation or other means known in the art.

The term “anti-sense nucleic acid molecule” as used in the context of the present invention is intended to encompass RNA or DNA and contain region(s) related to a specific target RNA transcript or its gene. It is also intended to include molecules giving rise to antisense sequences, including inverted repeat, sense RNA, etc.

The term “conditions permitting the RNAi factor to increase the degree to which the anti-sense nucleic acid molecule inhibits expression of the gene” is intended to include the concept of inhibition of gene expression which is high enough to be effective, but not so high as to harm the cell. The effective concentration range can be determined using routine methodology known in the art.

Reference to a “subject” is intended to include human, animal (mouse), plant, or other life forms.

Reference to a “cell” is intended to encompass eukaryotic cells such as yeast, mammalian, plant, etc

The term “disorder” will be understood to include viral infection (HIV, HPV, etc), cancer, autoimmune disease and other chronic and acute diseases.

The term “phenotype” as used in the context of the present invention is intended to include cell staining, morphology, growth, and similar characteristics.

### BRIEF DESCRIPTION OF THE FIGURES

FIG. 1. Target genes and antisense fragments. a, The strain KC4-6 expresses low levels of lacZ RNA. The lacZ expression cassette is integrated at the ura4 locus on chromosome III and is composed of the SV40 early promoter, the E. coli lacZ gene and the SV40 3′ processing signal. The 5′ and 3′ flanking DNA sequences of the ura4 locus are as indicated. b, The strain RB3-2, which expresses high levels of lacZ RNA, contains an expression cassette containing the S. pombe adh1 promoter, the lacZ coding region, and the S. pombe ura4 3′ processing signal. c, The full-length 3.5 kb antisense lacZ fragment is shown. The crippled sense fragment was generated by end-filling the ClaI cut lacZ vector and religating on itself. d, The AML1 strain contains a c-myc-lacZ fusion cassette integrated at the ura4 locus. Exons 2 and 3 of the human c-myc gene were employed as a target. The relative position of the c-myc antisense fragment is shown. Transcription initiation sites are indicated by the bent arrows. The straight arrows represent the normal direction of transcription for a particular DNA fragment.

FIG. 2. dsRNA-mediated gene silencing in fission yeast. a, A fission yeast strain containing the integrated lacZ gene under control of the strong adh1 promoter was transformed with lacZ antisense vectors. The antisense gene was under control of the weak nmt41 promoter (low antisense), the strong nmt1 promoter in single copy (15) (medium antisense), or the nmt1 promoter in multi-copy (high antisense). For the strain expressing maximum antisense RNA two episomal nmt1-driven antisense vectors containing different selectable markers were co-transformed into the target strain. b, A fission yeast strain containing the integrated lacZ gene under control of the weak SV40 promoter (low target) or strong adh1 promoter (high target) was transformed with the antisense lacZ vector or antisense lacZ and sense lacZ vectors. Strains were co-transformed with vectors containing appropriate selectable markers to complement auxotrophy. c, The lacZ inverted repeat vector was expressed in the high-expressing lacZ target strain. The strain expressing the antisense construct only is also indicated. d, A fission yeast strain containing an integrated c-myc-lacZ fusion gene was transformed with the sense construct, the antisense construct, or both. Antisense and sense constructs were also expressed in the high-expressing lacZ strain. All strains were transformed with appropriate control plasmids to complement auxotrophy.

FIG. 3. Effect of co-expressing single copies of sense and antisense genes. Strains containing target lacZ alone (RB3-2), the target and a single copy of the antisense gene (K40-7), and the target and both the antisense and frameshifted lacZ genes (M62-1) were assayed for β-galactosidase activity. pREP2 and pREP4 are parental plasmids containing the LEU2 and ura4 selectable markers respectively. Strains were transformed with the appropriate control plasmids to complement auxotrophy. For each sample three independent colonies were assayed in triplicate.

FIG. 4. RNA expression profiles in fission yeast strains a, The target strain RB3-2 was transformed with the lacZ inverted repeat vector (pM53-1) or co-transformed with antisense (pGT2) and sense (pM54-3) vectors and grown to mid-log in the absence of thiamine. Total RNA from each strain was separated on a 1% denaturing agarose gel, transferred to a nylon membrane and probed with the radioactively labeled 2.2 kb nmt1 fragment. The episomal lacZ signal was normalized with the endogenous nmt1 transcript and quantitated by phosphorimage analysis. The relative level of episomal lacZ expression is shown. b, The strain M38-1 which contains a single copy stably integrated lacZ inverted repeat gene was grown to mid-log in the absence of thiamine, its RNA isolated and analyzed by Northern hybridization. The relative steady-state level of the “panhandle” lacZ RNA is indicated in comparison to episomally expressed antisense lacZ RNA.

FIG. 5. In vivo dsRNA assay. a, The constructs employed for assaying the ability of the lacZ inverted repeat to form an intramolecular RNA duplex are shown. Each vector contains a functional lacZ fragment which gives the transformed strain a blue color phenotype when grown in the presence of X-GAL. The predicted secondary structure of the pM81-2 generated transcript is indicated. b, The strain 2037 was transformed with the vectors pM85-1, pM91-1 and pM81-2 in the absence of thiamine Single colonies were streaked on minimal media plates and overlayed with 0.5% agarose medium containing 500 μg/ml X-gal and 0.01% SDS.

FIG. 6. The effect of ded1 on dsRNA-mediated gene regulation. a, The ded1 gene was co-expressed in a fission yeast strain containing the integrated lacZ gene. β-galactosidase activity of the strain transformed with and without the antisense lacZ vector is shown. b, The target strain was transformed with a vector expressing a short lacZ antisense gene or with both the short lacZ antisense vector and the ded1-expressing vector. Strains were transformed with appropriate control plasmids to complement auxotrophy.

FIG. 7. Over-expression of thi1 in an antisense lacZ expressing strain. β-galactosidase activity of the target strain RB3-2 transformed with and without the antisense lacZ vector is shown. Over-expression of the thi1 gene in the antisense expressing strain is also indicated. Strains were transformed with appropriate control plasmids to complement auxotrophy.

FIG. 8. Over-expression screening strategy for RNAi modulating factors. A target strain containing the integrated lacZ gene under control of the adh1 promoter and the episomal vector containing the nmt1-driven lacZ antisense gene is transformed with an S. pombe cDNA library. Library fragments are driven by the nmt1 promoter. Transformants are individually screened for a change in the lacZ-encoded blue colony colour phenotype. These transformants are then quantitatively assayed for β-galactosidase activity in the presence and absence of thiamine. The antisense vector is then segregated out of transformants showing a cDNA dependent modification of lacZ suppression. A tertiary β-galactosidase assay is performed to determine if the effect is dependent on the presence of dsRNA. cDNA vectors are recovered from strains of interest, sequenced, and subjected to BLASTN analysis.

FIG. 9. Over-expression screen of a S. pombe cDNA library. a, Transformants were grown on minimal media plates and over-layed with X-GAL-containing medium. Those which showed a reduced blue colour-phenotype (black arrow) were analysed further. Transformants demonstrating an enhanced blue-colour phenotype were also identified (white arrow). b, Transformants which showed a visual reduction in the blue phenotype were assayed for β-galactosidase activity in liquid culture in the absence of thiamine. Thiamine was added to the medium to demonstrate that the enhanced lacZ suppression was dependent on RNA expression. Transformants were again assayed for β-galactosidase activity following antisense vector segregation. c, Over-expression of unique res genes in dsRNA-expressing strains.

FIG. 10. lacZ panhandle-mediated gene silencing. (A) The lacZ panhandle construct contains the full-length crippled lacZ gene which is followed by the inverted 5′ 2.5 kb lacZ fragment. Intramolecular hybridization generates an RNA with 2.5 kb RNA duplex and a 1 kb loop. (B) The relative steady-state level of the episomally expressed panhandle lacZ RNA (7.0 kb) is shown in comparison to episomally expressed antisense lacZ RNA (4.5 kb). The lacZ signals were normalized to the endogenous nmt1 transcript (1.3 kb) and quantitated by phosphorimage analysis. (C) The target strain was transformed with the episomally expressed lacZ panhandle and analyzed for b-galactosidase activity. The appropriate plasmids were co-introduced to complement auxotrophy. At least three independent colonies were assayed in triplicate for each strain. Transformants were assayed in the presence of thiamine to abrogate expression of the panhandle RNA (hatched). (D) The target lacZ strain was transformed with the panhandle vector only or both the panhandle and aes2 vector.

FIG. 11. Co-expression of ded1 and lacZ antisense genes. b-galactosidase activity of the target strain co-transformed with the ded1 gene and the antisense lacZ vectors is shown. Ded1 was expressed from the ura4-based plasmid pREP4 while the antisense genes were expressed from the LEU2-based plasmid pREP2. Strains expressing antisense RNAs or ded1 RNA alone are also indicated. Strains were transformed with appropriate control plasmids to complement auxotrophy. Three independent colonies were assayed in triplicate for each strain.

FIG. 12. DNA sequence for aes1 factor

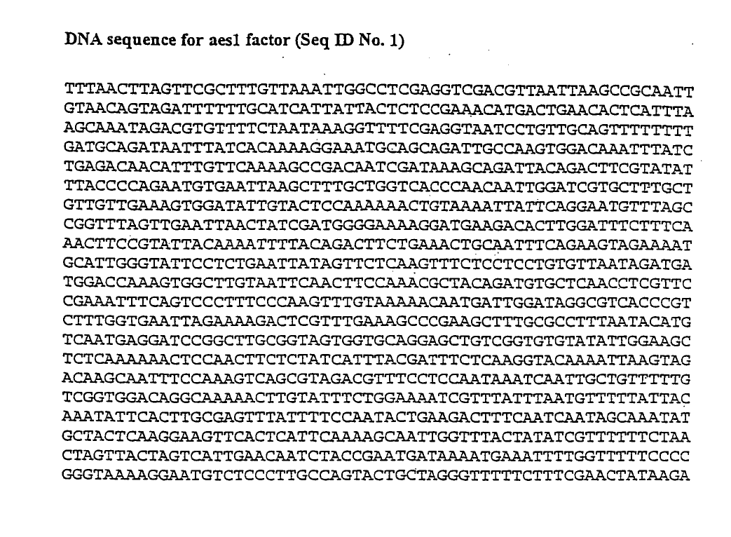

FIG. 13. DNA sequence for aes2 factor

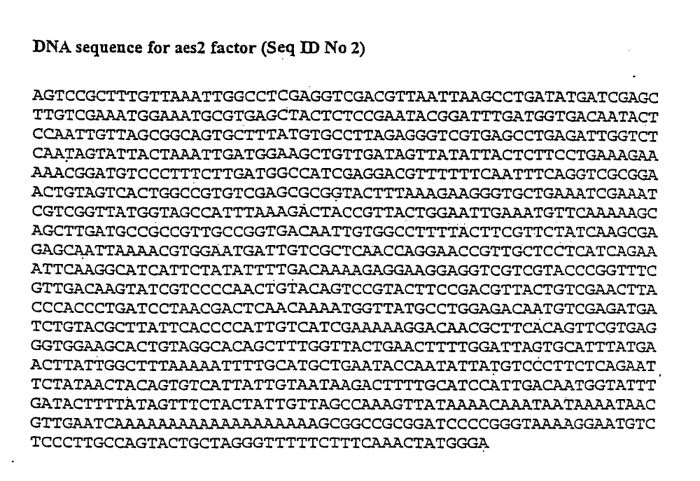

FIG. 14. DNA sequence for aes3 factor

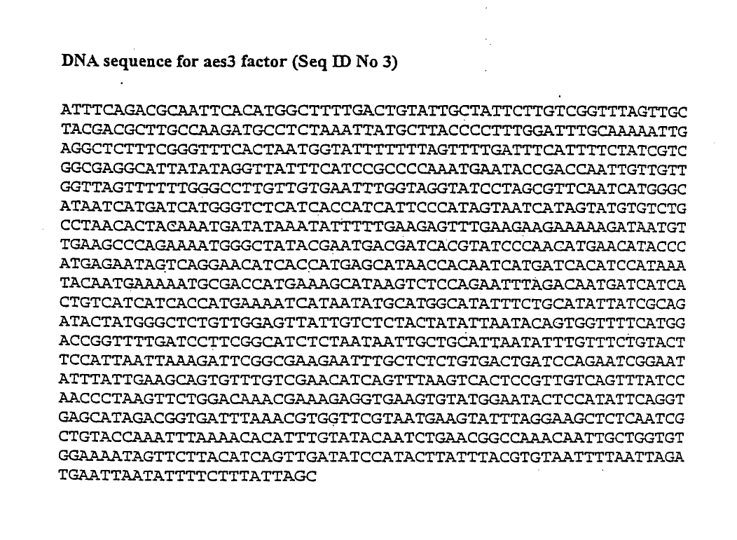

FIG. 15. DNA sequence for aes4 factor

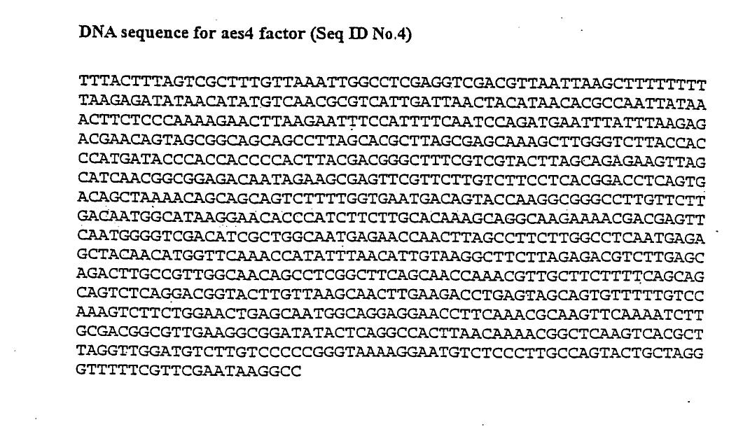

### DESCRIPTION OF THE PREFERRED EMBODIMENTS

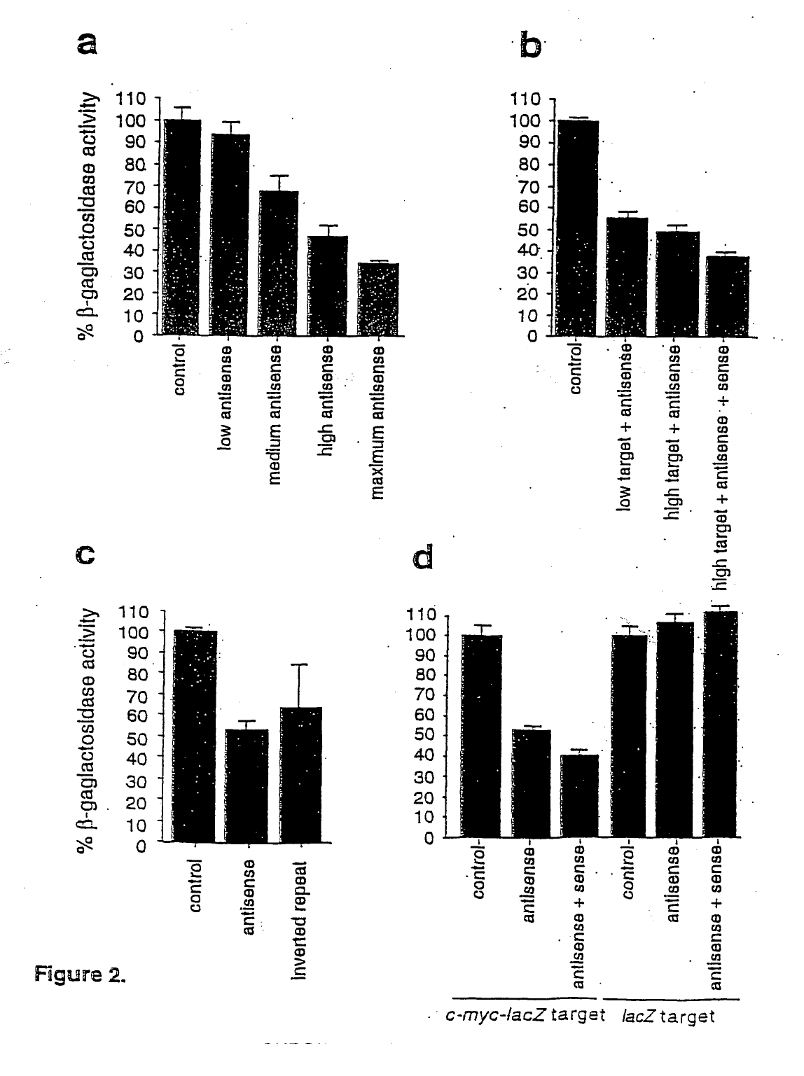

Using the lacZ fission yeast model to investigate features of antisense-RNA technology in vivo (1-4), it has been shown that the degree of target gene suppression is dependent on the level of antisense RNA (FIG. 2A)(1). During the course of developing this model it was surprisingly found that by increasing the steady-state levels of the lacZ target mRNA 20-fold in the presence of the same pool of antisense RNA that antisense-mediated suppression was not only maintained, but enhanced (FIG. 2B). To verify that this effect was independent of the target mRNA a lacZ gene containing a frameshift mutation (leading to an inability to translate the β-galactosidase enzyme) was co-expressed in antisense-expressing strains. Again, additional stimulation of lacZ inhibition was observed (FIG. 2B). These observations are consistent with the potential formation of additional dsRNA and consequent stimulation of gene silencing. They also indicate that the dsRNA-mediated gene silencing is dose-dependent in this system. To further show that dsRNA was the key component in target gene inhibition in fission yeast dsRNA was generated by expressing a lacZ inverted repeat with 2.5 kb of internal complementarity in the presence of lacZ target mRNA. Expression of this construct also reduced β-galactosidase activity in a lacZ expressing strain to a similar level to the full-length antisense RNA (FIG. 2C). Overall, these data suggested that increasing the potential formation of dsRNA, but not necessarily an antisense RNA:target mRNA hybrid, resulted in efficient interference of target gene expression in S. pombe.

To test the ability of dsRNA to specifically interfere with another target sequence in fission yeast, sense and antisense c-myc sequences were co-expressed in a strain containing an integrated c-myc-lacZ fusion cassette (3). A 792 bp antisense c-myc fragment from exon 2 of the human c-myc gene was previously found to suppress β-galactosidase activity within the c-myc-lacZ fusion target strain by 47% (3). β-galactosidase assays demonstrated that co-expression of the antisense and sense c-myc constructs in the target strain enhanced c-myc suppression compared with the antisense c-myc vector alone (FIG. 2D). Transformation of the strain expressing only the lacZ target with the antisense and sense c-myc constructs resulted in no down-regulation of β-galactosidase activity indicating that the action of dsRNA is sequence-specific (FIG. 2D). All of the above observations, as well as the reduction in target mRNA levels in response to additional dsRNA (data not shown), are consistent with features associated with RNAi. This is the first example of dsRNA-mediated gene regulation in yeast and, unlike RNAi in all other organisms except zebrafish, appears to be dependent on the concentration of intracellular dsRNA. The variation in RNAi efficacy between organisms may be due to the absence or under-expression of some components of the RNAi pathway or the presence of RNAi inhibitors. We have now further developed this genetic model for identification of factors involved in RNAi.

Without wishing to be bound by any particular mechanism, it is probable that a multi-protein complex exists which mediates the post-transcriptional degradation of target mRNA in RNAi. Several genes which are involved in the RNAi phenomenon have been identified through genetic screens in Neurospora and nematodes. These genes include an RNA-dependent RNA polymerase (qde-1, ego-1) a RecQ DNA helicase (qde-3), an RNase D homologue (mut-7), and a putative translation initiation factor (rde-1, qde-2). The genes mut-2, rde-2, rde-4, and rde-7 have also been identified as being involved in either the initiation or maintenance of RNAi. Additionally, Drosophila cell free assays have shown that RNAi is mediated by nuclease degradation of the targeted mRNA while a 21-25 nt RNA species appears to be integral to specific post-transcriptional genetic interference. RNAi has also been shown to be dependent on ATP which may be required for strand dissociation of dsRNA. However, a missing component of the proposed multi-protein complex in the RNAi pathway is the implied RNA helicase.

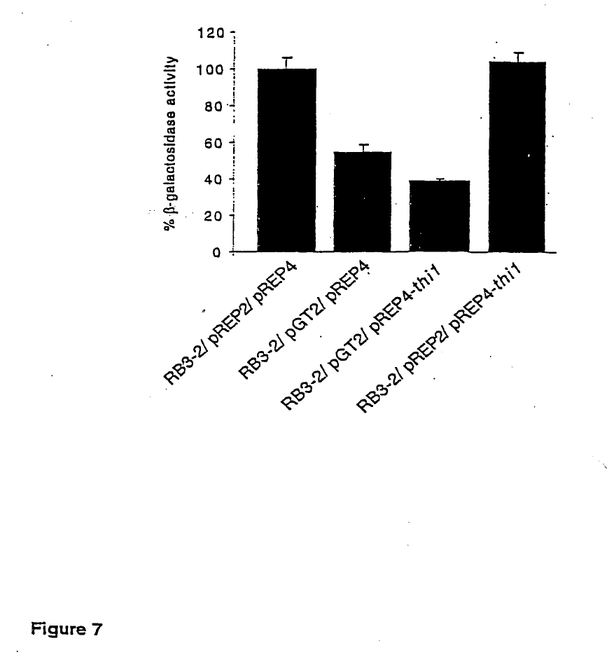

The dose-dependency of dsRNA-mediated gene silencing in fission yeast described above has allowed us to use an over-expression strategy to identify genes involved in RNAi. In comparison to mutagenesis strategies, over-expression can enable the identification of genes which are otherwise essential for cell viability. Also, cellular factors that quantitatively enhance or reduce RNAi activity can be determined. The first gene that was tested in mediating RNAi activity in the present model was the nmt1 transcription factor thi1. This gene has been shown to specifically up regulate nmt1 expression when overexpressed in fission yeast. As we have previously shown that antisense RNA-mediated gene suppression is dose dependent in S. pombe (1) it was hypothesized that over-expression of thi1 would result in increased production of nmt1-driven antisense lacZ RNA and a consequent enhancement in target gene suppression. The thi1 open reading frame was PCR amplified and subcloned into pREP4 as a BamHI fragment. This vector was then transformed into RB3-2/pGT2 and β-galactosidase assays performed. As predicted, lacZ suppression was enhanced when compared to a strain expressing antisense RNA alone (FIG. 7). This result indicated that host-encoded factors are present which affect the robustness of dsRNA-mediated gene suppression in fission yeast and also validated the overexpression strategy.

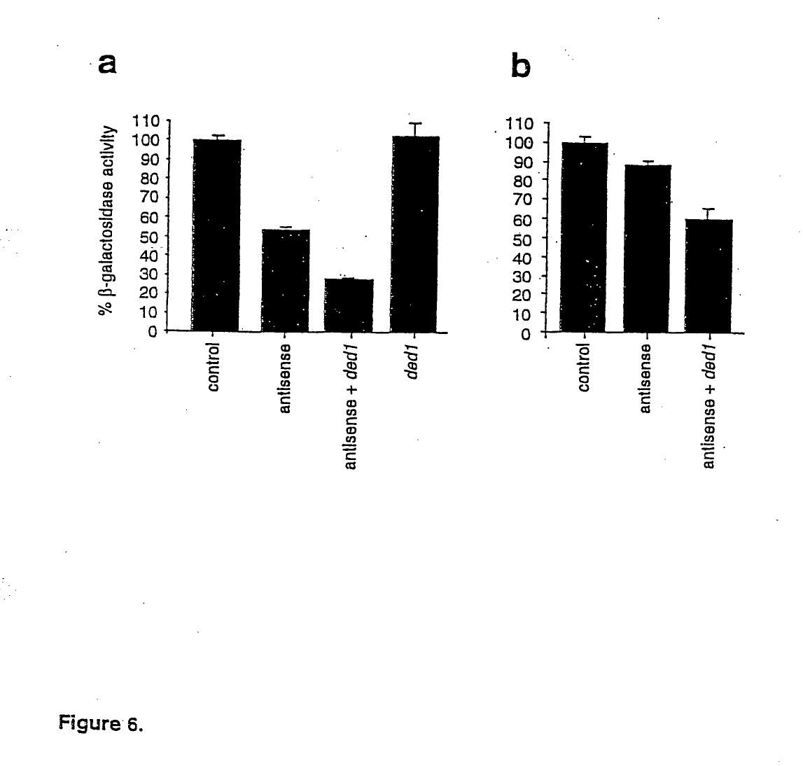

The second gene investigated has been the S. pombe ATP-dependent RNA helicase gene, ded1. Ded1 is an essential gene which has previously been characterized as a suppressor of sterility, a suppressor of checkpoint and stress response, and a general translation initiation factor. According to current models of dsRNA-mediated gene regulation an ATP-dependent RNA helicase may be required in conjunction with a dsRNA-dependent RNA polymerase for the formation of short single-stranded RNA fragments which specifically degrades target mRNA. It was therefore reasoned that over-expression of this gene in fission yeast could enhance the efficiency of dsRNA-mediated gene silencing by stimulating the unwinding of dsRNA. Co-expression of the ded1 vector with the antisense lacZ vector significantly enhanced dsRNA-mediated lacZ inhibition by a further 50% compared to the antisense expressing strain (FIG. 6A). When ded1 was over-expressed in the absence of the antisense lacZ vector, β-galactosidase activity was comparable to control strains indicating that its effect was dependent on the presence of dsRNA (FIG. 6A). The ded1 vector was also co-expressed with a short antisense lacZ vector which has previously been shown to be less effective than the full-length antisense gene (16). Again, the over-expression of ded1 stimulated dsRNA-mediated lacZ inhibition (FIG. 6B). It was thus shown that over-expression of an ATP-dependent RNA helicase greatly enhances RNAi in fission yeast.

By over-expressing a cDNA library in a dsRNA fission yeast model we have identified four novel, genes with potential roles in the RNAi pathway. All of the known genes identified are essential and have natural associations with DNA, RNA or nucleic acid binding proteins, consistent with the expectation of RNAi cellular factors. In addition, two of the three known cDNAs encoded proteins involved in the process of translation. These findings along with our previous identification of an ATP-dependent RNA helicase (Raponi & Arndt, submitted) and reports of the involvement of a translation initiation factor in RNAi and the co-purification of ribonuclease activity with ribosomal fractions, suggests that this may be one site at which RNAi functions. Alternatively, the identification of such varied proteins as DNA helicases and translational components as part of the RNAi machinery implies that particular cellular proteins may be recruited into more than one multiprotein complex. Under the latter conditions, over-expression of these specific proteins may result in the generation of an RNAi complex that recognises dsRNA and mediates target gene suppression. Certain of these proteins may be rate-limiting or rate-determining in RNAi and only through supplementation of these factors are their roles in RNAi uncovered. It is proposed, without wishing to be bound by any particular mechanism, that in addition to the previously identified core proteins in the RNAi multiprotein complex(es), additional factors such as EF Tu, L7a, sna41 and res1 may be enlisted for a role in dsRNA-mediated gene suppression.

The over-expression strategy described overcomes some limitations associated with mutagenesis by identifying essential genes with a role in RNAi. In addition, this strategy complements these other systems by allowing the isolation of cellular factors that modify the efficacy of RNAi in vivo. The roles of these modulators of RNAi may be varied and include recognition and amplification of the dsRNA, delivery of the small 21-25 nt dsRNAs to the target mRNA, association between the antisense and target mRNA strands, and RNAi complex formation. Alternatively, these modulators may control the rate of RNAi or the formation of different complexes within cell types or for different forms of post-transcriptional gene silencing. The fact that over-expression of specific RNAi modulators enhanced dsRNA-mediated gene regulation in fission yeast indicates that a similar approach could be used to identify RNAi modulators in other organisms. In addition, the co-expression of these factors with different forms of post-transcriptional gene silencing including co-suppression, quelling, and antisense RNA could be one way of enhancing the efficacy of these methods. This may be especially important for application of RNAi to mammalian cells and tissues or to genes which have been somewhat recalcitrant to this form of regulation.

The present invention demonstrates for the first time the intrinsic involvement of an ATP-dependent RNA helicase as a key component in RNAi. Further, it can be rate limiting, as the over-expression leads to increased RNAi activity in this system. This ability of the ded1-encoded RNA helicase is consistent with its activities as a member of the DEAD box family of helicases, with their three core domains of ATPase, RNA helicase, and RNA binding activities. These could allow the enzyme to enhance gene suppression as follows: (i) in a dissociative mechanism it could mediate either the unwinding of dsRNA to generate a cRNA in conjunction with an RNA-dependent RNA polymerase or strand separation of fragmented dsRNA to enhance binding to homologous transcripts, and/or (ii) in an associative mechanism it could catalyse the ATP-dependent exchange of the sense strand of the short dsRNA with the target mRNA. The presence of ded1 homologues in other organisms displaying RNAi further supports the general involvement of this component in the RNAi machinery.

The present invention also provides for the first time a novel and quantitative genetic system based on S.pombe, for rapidly identifying essential cellular factors involved in RNAi. The use of this model has enabled verification of RNA helicase activity as a critical contributor to efficient RNAi activity and isolated novel RNAi factors including EF Tu, L7a, sna41, and an unidentified gene res1. The present invention also demonstrates that gene silencing may be enhanced by concomitant expression of such RNAi factors.

The invention will now be described more particularly with reference to non-limiting examples of certain preferred embodiments of the invention.

### Example 1

### Materials and Methods Used to Exemplify the Invention

S. pombe Media and Manipulations.

All yeast strains were maintained on standard YES or EMM media (6). Repression of nmt1 transcription was achieved by the addition of thiamine to EMM media at a final concentration of 4 μM (7). Yeast cells were transformed with plasmid DNA by electroporation (8) and stable integrants were identified as previously described (6). A glass bead procedure (9) was used to isolate genomic DNA which was used for Southern analysis and PCR diagnosis. Total RNA was extracted as previously described (10).

S. pombe Strain and Plasmid Construction.

Construction of the low expressing lacZ strain, KC4-6 (h⁻, ura4::SV40-lacZ, leu1-32), has been previously described (2). The strain RB3-2 (h⁻, ura4::adh1-lacZ, leu1-32) which expresses higher levels β-galactosidase has also been previously described (1). The target strain which contains the c-myc:lacZ fusion has previously been described (3).

The construction of the long lacZ antisense containing episomal plasmid, pGT2, and corresponding control plasmids have been described (2). Plasmid pREP4-As was generated by subcloning the lacZ BamHI fragment contained in pGT2 into the plasmid pREP4 (11). pREP4 is identical to pREP1 except that the S. cerevisiae LEU2 gene has been replaced with the S. pombe ura4 selectable marker. To decrease the steady-state level of episomally expressed antisense lacZ, plasmids pREP42-As and pREP82-As were constructed by subcloning the lacZ BamHI fragment from pGT2 into pREP42 and pREP82, respectively. These plasmids are derivatives of pREP4 with mutations in the TATA box of the nmt1 promoter (12). The crippled lacZ vector, pGT62, was generated by end-filling the ClaI site of pGT2 and re-ligating (2). This frameshifted fragment was then subcloned into the BamHI site of pREP4 to generate the plasmid pM54-3.

The lacZ panhandle integration vector, pM30-8, was generated by first introducing a NotI site into the XmaI site of pRIP1/s (11) using the self-complementary linker 5′ CCG GGC GGC CGC 3′ to generate pL121-14. The 2.5 kb sequence of the 5′ end of the frameshifted lacZ gene (2) was then PCR-amplified to give it NotI ends using the forward primer 5′ ATGCGGCCGCAATTCCCGGGGATCGAAAGA 3′ and reverse primer 5′ ATGCGGCCGCAATGGGGTCGCTTCACTTA 3′. This product was cloned using a TA cloning kit (TOPO: Invitrogen, San Diego, Calif., USA) and then subcloned into the NotI site of pL121-14 in the antisense orientation. The integrating vector was introduced into target strains in single copy by using the sup3-5/ade6-704 complementation system (13). The full-length frameshifted lacZ fragment was then introduced into the BamHI site of this vector in the sense orientation upstream of the 2.5 kb antisense fragment to generate pM30-8. The episomal version of this vector (pM53-1) was made by removing the Pst1 sup3-5 fragment and introducing the autonomous replicating sequence as an EcoRI fragment (11). For testing the ability of the panhandle construct to form dsRNA in vivo, the frameshifted lacZ fragment was replaced with the functional lacZ fragment in the vectors pM54-3 and pM53-1, to generate the vectors pM85-1 and pM81-2, respectively. The vector pM91-1, which is unable to form a lacZ panhandle transcript, was generated by removing the 2.5 kb NotI lacZ fragment from pM81-2 and reintroducing it in the sense orientation.

Generation of the c-myc antisense construct, pCM-17, has been described elsewhere (3). The 792 by BglII c-myc fragment from pCM-17 was subcloned into the BamHI site of pREP4 in the sense orientation to generate pN12-1.

The ded1, and thi1, open reading frames were amplified from fission yeast genomic DNA (strain 1913). ded1 was amplified to give it BamHI ends using the forward primer 5′ ATGGGATCCCAACCAAACACTTCAACTCAG 3′ and the reverse primer 5′ ATGGGATCCTCAGAAGCCTGTGCATAACAC 3′. thi1 was amplified to give it BglII ends using the forward primer 5′ ATGAGATCTGTGGTTGGTATTCTAGAGAGA 3′ and the reverse primer 5′ ATGAGATCTAACAAAGACCTGCAAAAAACC 3′. PCR products were purified (Qiagen PCR purification kit), digested with either BamHI or BglII, gel-purified (Qiagen), and subcloned into the BamHI site of pREP4 in the sense orientation.

### Southern and Northern Analysis.

Nucleic acid electrophoresis and membrane transfer was performed as described (14). Southern and Northern blots were hybridized using ExpressHyb solution according to the manufacturer's instructions (Clontech Laboratories). DNA probes were ³²P-labelled using the Megaprime labelling kit (Amersham). Probes included a 960 bp BamHI/ClaI lacZ fragment from pI2-1 (1), a 570 by HindIII/EcoRI ura4-3′ fragment from pGT113 (15), and a 2.2 kb PstI/SacI nmt1 fragment from pRIP1/s. Radioactive signals were detected by autoradiography and quantitated by phosphorimage analysis (ImageQuant; Molecular Dynamics).

### Plasmid Distribution Analysis.

Plasmid co-transformants of strain RB3-2 were grown under selective conditions to a cell density of 1-2×10⁷ cells/ml. A serial dilution of each culture was performed and cells plated for single colonies in triplicate onto each of YES, EMM, EMM+leucine, and EMM+uracil agar media. The number of colonies grown on YES was taken as the total number of viable cells, while colonies growing on EMM represented the cells in the sampled population that contained both the ura4-containing (pREP4-based) and the LEU2-containing (pREP1-based) plasmids. Cells containing either the ura4-containing plasmid or the LEU2-containing plasmid were identified from the EMM+leucine and EMM+uracil plates respectively. The ratio of the number of colonies grown on selective media to the total number of viable colonies was used as the quantitative measure of the proportion of cells in the population, grown under selection, which contained plasmids.

β-galactosidase Assays.

The expression of the lacZ gene-encoded product, β-galactosidase, was quantitated using a cell permeabilization protocol as previously described (Raponi et al., 2000). A semi-quantitative overlay assay was also employed for rapid screening of yeast transformants (3).

Isolation of S. pombe cDNA Clones That Alter Antisense RNA Efficacy.

The S. pombe cDNA library was originally constructed in pREP3Xho by Bruce Edgar and Chris Norbury (5). The vector pREP3Xho is derived from pREP3 which contains the LEU2 marker and inserts are under control of the nmt1 promoter (11). A total of 5 μg of library DNA was transformed into the strain RB3-2 containing the episomal antisense lacZ vector pREP4-lacZAS and grown in EMM liquid media to the mid-logarithmic phase. Transformants were then plated on EMM solid media and grown at 30° C. for 3 days. Colonies were over-layed with medium containing 0.5 M sodium phosphate, 0.5% agarose, 2% dimethylformamide, 0.01% SDS, and 500 μg/ml X-GAL (Progen, Australia). Plates were then incubated at 37° C. for 3 hrs, colonies of interest recovered and assayed for β-galactosidase activity.

### Plasmid Segregation.

Strains were plated on EMM containing limiting uracil and 1 mg/ml 5-fluoroorotic acid (6). Strains were then replica plated on both selective and non-selective media. Those colonies that did not grow on selective media were identified as having lost the ura4-containing antisense plasmid. To determine the mitotic stability of pREP-based plasmids we used the plasmid segregation method previously described (16). This method indicated that up to 30% of the cells grown in selective media did not contain the resident plasmid.

### Example 2

### Effect of Steady-State Level of Target mRNA and Antisense RNA on Antisense Efficacy

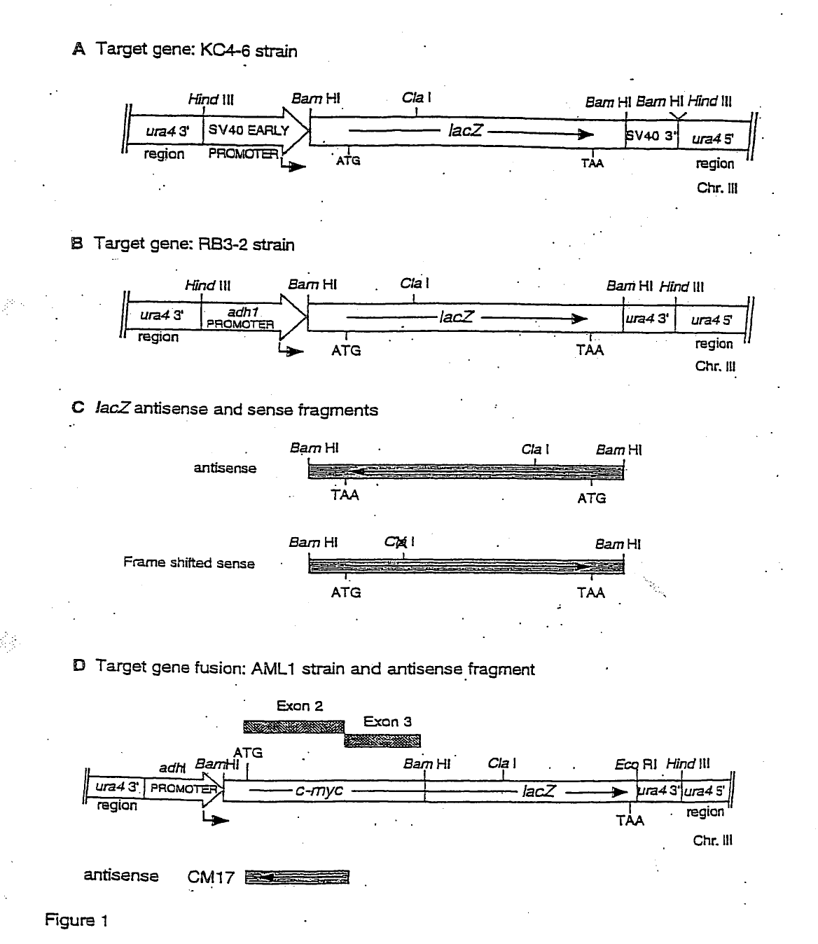

To determine the role of target mRNA steady-state levels in antisense-mediated gene suppression, we investigated the ability of a long lacZ antisense RNA (2) to regulate lacZ target genes under control of both weak and strong constitutive promoters. The low-level expressing strain, KC4-6, contained the lacZ gene driven by the SV40 early promoter integrated at the ura4 locus in chromosome III (FIG. 1A; (2)). The high-level lacZ expressing strain, RB3-2, was constructed by placing the lacZ gene under control of the strong fission yeast adh1 promoter and the 3′ processing signal from the ura4 gene and integrating this expression cassette at the ura4 locus (FIG. 1B; (1)). Characterization of both strains indicated that RB3-2 expressed 20-fold more lacZ mRNA than KC4-6 while an approximate 40-fold increase in β-galactosidase was also detected (data not shown). Both strains were transformed with the lacZ antisense gene-containing plasmid (pGT2) and its corresponding sense control plasmid (pGT62). β-galactosidase assays indicated that the antisense RNA suppressed β-galactosidase activity by 45% in KC4-6 while the same RNA reduced β-galactosidase activity by 52% in RB3-2 (FIG. 2B). No reduction in β-galactosidase activity was seen in either strain when transformed with the control plasmid pGT62 which expresses a crippled version of lacZ. These results indicated that despite a 20-fold increase in the steady state level of the lacZ target mRNA in RB3-2 the efficacy of antisense-mediated down regulation was not only maintained, but enhanced. The increase in gene suppression demonstrated with strain RB3-2 was also seen when antisense molecules targeted to subregions of the lacZ target gene were used, but was not as pronounced. These data suggested an increase in target mRNA can result in enhanced target gene suppression.

It has previously, been demonstrated that a stably integrated antisense lacZ gene acts in a dose dependent fashion with the steady-state levels of antisense RNA being dependent on genomic position effects and transgene copy number (1). Here the role of the steady-state level of episomally expressed antisense RNA in both the low lacZ expressing target strain, KC4-6, and the high lacZ expressing target strain, RB3-2, was investigated. The expression of lacZ antisense RNA was increased by co-transforming KC4-6 and RB3-2 with plasmids pGT2 and pREP4-As, each of which contains the lacZ antisense gene under control of the nmt1 promoter, but different selectable markers. To decrease the steady-state level of antisense RNA, the plasmids pREP42-As and pREP82-AS were employed. The nmt1 promoter in these vectors contain deletions in the TATA box sequence which affect the level of transcription, but have no impact on the site of transcription initiation or thiamine repressibility (12). Each of the different antisense gene-containing plasmids was co-transformed with a control plasmid to complement auxotrophy where appropriate. This resulted in a set of co-transformants of both RB3-2 and KC4-6 each containing the same lacZ antisense gene, but with different promoter capacities. Each co-transformant was analyzed for antisense RNA steady-state levels and β-galactosidase activity. Table 1 indicates that with both strains the degree of target suppression is enhanced with the increase of antisense lacZ RNA expression.

TABLE 1
Effects of varying the level of antisense RNA in strains
expressing both low and high level target.

| | | | | | Relative |
|---|---|---|---|---|---|
| | | | β-gal. activity | | antisense |
| Strain | Plasmids | Descriptions | (units) | % suppression^{a} | RNA level^{b} |
| KC4-6 | pREP2, pREP4 | Vector | 1.07 | 0 | 0 |
| | pREP2, pREP82-AS | Low As | 1.10 | 0 | 0.04 |
| | pREP2, pREP42-AS | Medium As | 1.07 | 0.1 | 0.14 |
| | pREP2, pREP4-AS | Full As-ura4 | 0.75 | 30 | 1.0 |
| | pGT2, pREP4 | Full As-LEU2 | 0.70 | 34.6 | 2.2 |
| | pGT2, pREP4-AS | Double-As | 0.54 | 49.2 | 5.6 |
| RB3-2 | pREP2, pREP4 | Vector | 48.5 | 0 | 0 |
| | pREP2, pREP82-AS | Low As | 44.0 | 9.4 | 0.05 |
| | pREP2, pREP42-AS | Medium As | 43.9 | 9.6 | 0.18 |
| | pREP2, pREP4-AS | Full As-ura4 | 27.2 | 43.9 | 1.0 |
| | pGT2, pREP4 | Full As-LEU2 | 24.9 | 48.7 | 1.9 |
| | pGT2, pREP4-AS | Double-As | 16.6 | 65.8 | 5.4 |

^{a}The % suppression of β-galactosidase activity was determined by expressing each β-galactosidase activity as a percentage of the activity found in the pREP2, pREP4 co-transformant.
^{b}Within each strain the steady-state level of long lacZ antisense RNA was normalized to nmt1 mRNA and then expressed relative to the level observed in the pREP2, pREP4-As co-transformant.

These data agree with the previous report that antisense RNA-mediated gene inhibition is dose dependent in S. pombe (1). However, for each co-transformant the degree of β-galactosidase suppression was greater by 10-15% in the high lacZ expressing strain, RB3-2, than the low lacZ expressing strain, KC4-6, again demonstrating that low levels of target mRNA can limit antisense efficacy.

### Example 3

### Effect of Increasing Sense RNA on Antisense RNA-Mediated Target Gene Suppression

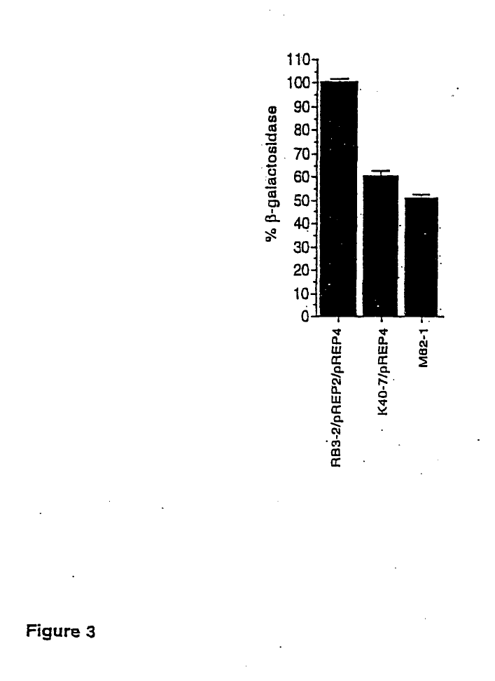

To determine whether the increase in gene suppression was due to formation of an antisense RNA:target mRNA hybrid or an antisense RNA:sense RNA hybrid, a version of lacZ which is unable to be translated into functional β-galactosidase was co-expressed in strains expressing the lacZ antisense gene. If antisense RNA was required to hybridize to target mRNA for inhibition of the gene expression pathway, then over-expression of the sense RNA would compete with the target for the available antisense molecules and a decrease in lacZ gene suppression would result. Initially, both antisense and sense lacZ vectors were integrated in single copy into separate target strains and then crossed with each other. In strains containing the antisense gene alone, β-galactosidase activity was reduced by approximately 40% (FIG. 3; (1)) while there was no reduction in the strain expressing sense lacZ alone (data not shown). However, in the strain expressing both complementary transcripts (M62-1), β-galactosidase activity was reduced by 50% (FIG. 3). These results suggested that increasing the intracellular concentration of sense RNA in the presence of antisense RNA, and therefore potentially generating more dsRNA, stimulates target gene suppression.

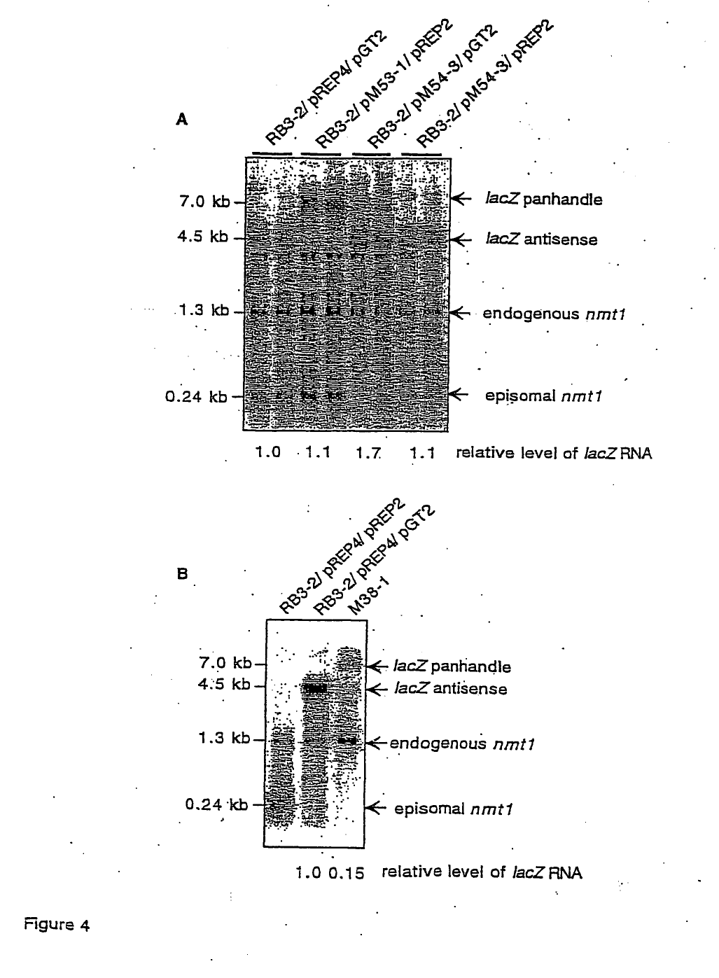

To further increase the potential formation of intracellular dsRNA an episomal sense lacZ plasmid (pM54-3; FIG. 2B) was co-transformed with the episomal antisense lacZ plasmid, pGT2, into the target strain R133-2. Both of these sequences were expressed by the strong conditional nmt1 promoter and RNA analysis showed that each transcript was being expressed at high levels when grown in the absence of thiamine (FIG. 4A). When both RNAs were co-expressed in RB3-2, the target lacZ was suppressed by 65% compared to 50% in the strain expressing the antisense plasmid alone (FIG. 2B). Northern analysis demonstrated that the total relative level of episomally expressed nmt1-driven transgenes was approximately 60% higher than when either vector was expressed alone (FIG. 4A). Although expression of these complementary RNA transcripts is at a high level compared with single copy integrants only an additional 15% suppression was observed.

One explanation for the absence of higher levels of target gene suppression is plasmid segregation. S. pombe undergoes asymmetric segregation, with the result that mitosis produces a daughter cell which lacks the segregating plasmid (17). Therefore, when the 65% suppression level seen in the total population is corrected for only those cells containing the ura4-based and LEU2-based plasmids the level of suppression approaches approximately 100% (data not shown). Overall these data suggest that increasing the potential formation of dsRNA, but not necessarily an antisense RNA:target mRNA hybrid, is required for efficient interference of target gene expression in S. pombe. However, unlike the phenomenon of RNAi seen in plants and nematodes, the dsRNA-mediated gene suppression demonstrated in fission yeast seems to be dependent on the concentration of intracellular dsRNA or a threshold level of dsRNA is required to invoke potent gene silencing.

### Example 4

### Effect of a lacZ Inverted Repeat on lacZ Gene Expression

To further investigate the ability of dsRNA to inhibit gene expression in fission yeast we generated a vector containing the full-length 3.5 kb frameshifted lacZ sequence with a 2.5 kb inverted repeat. This construct generates a transcript of approximately 7 kb in length with 2.5 kb of self-complementarity which, predictably, will form a strong intramolecular RNA duplex. This gene was initially integrated into a fission yeast strain in single copy and then crossed with the strain RB3-2. The resulting strain which contained the single copy inverted repeat gene and target lacZ showed no reduction in β-galactosidase activity when transcription of the inverted repeat was activated. Southern analysis confirmed that the cassette was intact (data not shown) while RNA analysis indicated that the 7 kb transcript was being generated but approximately 10-fold less than episomally expressed antisense lacZ (FIG. 4A&B). To increase the steady-state level of the inverted repeat RNA we generated the episomal plasmid pM53-1 containing this cassette. RNA analysis demonstrated that this vector is expressed at a similar level to episomally expressed antisense lacZ (FIG. 4A). When expressed in RB3-2 pM53-1 inhibited β-galactosidase activity by 40% (FIG. 2C), while addition of thiamine to the culture medium returned β-galactosidase activity to control level.

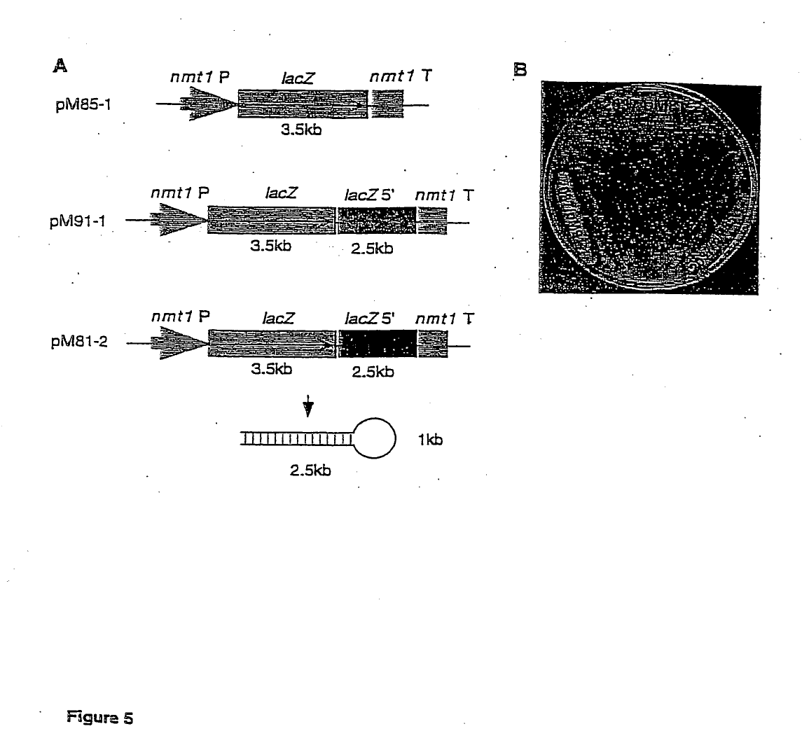

To confirm that gene inhibition was due to this construct forming an RNA duplex, an in vivo assay for dsRNA was developed. To this end, a series of vectors were generated which contained functional lacZ sequences including lacZ alone (pM85-1), a lacZ inverted repeat (pM81-2), and the lacZ repeat with both sequences in the sense orientation (pM91-1) (FIG. 5A). These vectors were then introduced into a strain lacking the integrated target sequence (NCYC2037; h⁺, ura4-D18) and resulting transformants were overlayed with X-GAL-containing agarose. Both strains containing control vectors generated high levels of β-galactosidase while the strain expressing the lacZ inverted repeat did not (FIG. 5B). In each case Northern analysis demonstrated that strains were expressing the transgene. These results indicate that the inverted repeat RNA could not be efficiently translated to produce β-galactosidase, most probably due to the formation of a strong intramolecular hairpin structure. Taken together, these results show that a construct which has the ability to form dsRNA by intramolecular hybridization will interfere with target gene expression when expressed at high levels, again indicating that dsRNA mediates target gene suppression in a dose-dependent manner in fission yeast.

### Example 5

### Gene-Specificity of dsRNA-Meditated Gene Silencing in Fission Yeast

To test the ability of dsRNA to specifically interfere with other target sequences in fission yeast, sense and antisense c-myc sequences were co-expressed in a strain containing an integrated c-myc-lacZ fusion cassette (FIG. 1D; (3)). A 792 bp antisense c-myc fragment from exon 2 of the human c-myc gene (named CM-17) was previously found to suppress β-galactosidase activity within a c-myc-lacZ fusion target strain (AML1) by 47% (3). CM-17 was subsequently subcloned into pREP4 in the sense orientation to generate pN12-1. The antisense c-myc vector (pCM-17) and the sense c-myc vector were transformed into AML1 both independently and together. β-galactosidase assays demonstrated that co-expression of the antisense and sense constructs enhanced c-myc suppression by an additional 13% compared with the antisense c-myc vector alone (FIG. 2D). Transformation of RB3-2 with the antisense and sense c-myc constructs resulted in no down-regulation of β-galactosidase activity indicating that the action of dsRNA is gene-specific (FIG. 2D). These results demonstrate that dsRNA can specifically interfere with multiple targets in fission yeast including one of human origin.

### Example 6

### Over-Expression of a Translation Factor can Enhance RNAi in Fission Yeast

The dose-dependency of dsRNA-mediated gene silencing in fission yeast described above has allowed us to use an over-expression strategy to identify genes involved in RNAi. In comparison to mutagenesis strategies, over-expression can enable the identification of genes which are otherwise essential for cell viability. Also, cellular factors that quantitatively enhance or reduce RNAi activity can be determined. The first gene that we have tested in mediating RNAi activity in the present model has been the S. pombe ATP-dependent RNA helicase gene, ded1. Ded1 is an essential gene which has previously been characterized as a suppressor of sterility, a suppressor of checkpoint and stress response, and a general translation initiation factor. According to current models of dsRNA-mediated gene regulation an ATP-dependent RNA helicase may be required in conjunction with a dsRNA-dependent RNA polymerase for the formation of short single-stranded RNA fragments which specifically degrades target mRNA. We therefore reasoned that over-expression of this gene in fission yeast could enhance the efficiency of dsRNA-mediated gene silencing by stimulating the unwinding of dsRNA. Co-expression of the ded1 vector with the antisense lacZ vector significantly enhanced dsRNA-mediated lacZ inhibition by a further 50% compared to the antisense expressing strain (FIG. 6A). When ded1 was over-expressed in the absence of the antisense lacZ vector, β-galactosidase activity was comparable to control strains indicating that its effect was dependent on the presence of dsRNA (FIG. 6A). The ded1 vector was also co-expressed with a short antisense lacZ vector which has previously been shown to be less effective than the full-length antisense gene (2). Again, the over-expression of ded1 stimulated dsRNA-mediated lacZ inhibition (FIG. 6B). We have thus shown that over-expression of an ATP-dependent RNA helicase greatly enhances RNAi in fission yeast. Although an ATP-dependent RNA helicase has recently been suggested to be a key component in RNAi, this is the first demonstration of its intrinsic involvement. Further, it can clearly be rate limiting, as the over-expression leads to increased RNAi activity in this system. This ability of the ded1-encoded RNA helicase is consistent with its activities as a member of the DEAD box family of helicases, with their three core domains of ATPase, RNA helicase, and RNA binding activities. These could allow the enzyme to enhance gene suppression as follows: (i) in a dissociative mechanism it could mediate either the unwinding of dsRNA to generate a cRNA in conjunction with an RNA-dependent RNA polymerase or strand separation of fragmented dsRNA to enhance binding to homologous transcripts, and/or (ii) in an associative mechanism it could catalyse the ATP-dependent exchange of the sense strand of the short dsRNA with the target mRNA. The presence of ded1 homologues in other organisms displaying RNAi further supports the general involvement of this component in the RNAi machinery.

### Example 7

### Over-Expression of a Transcription Factor can Enhance RNAi in Fission Yeast

The second gene investigated was the nmt1 transcription factor thi1. This gene has been shown to specifically up regulate nmt1 expression when overexpressed in fission yeast. As we have previously shown that antisense RNA-mediated gene suppression is dose-dependent in S. pombe (1) it was hypothesized that over-expression of thi1 would result in increased production of nmt1-driven antisense lacZ RNA and a consequent enhancement in target gene suppression. The thi1 open reading frame was PCR-amplified and subcloned into pREP4 as a BamHI fragment. This vector was then transformed into RB3-2/pGT2 and β-galactosidase assays performed. As predicted, lacZ suppression was enhanced when compared to a strain expressing antisense RNA alone (FIG. 7). This result indicated that host-encoded factors are present which modulate the efficacy of dsRNA-mediated gene suppression in fission yeast and also validated the over-expression strategy.

### Example 8

### Screening for Novel RNAi Modulating Factors

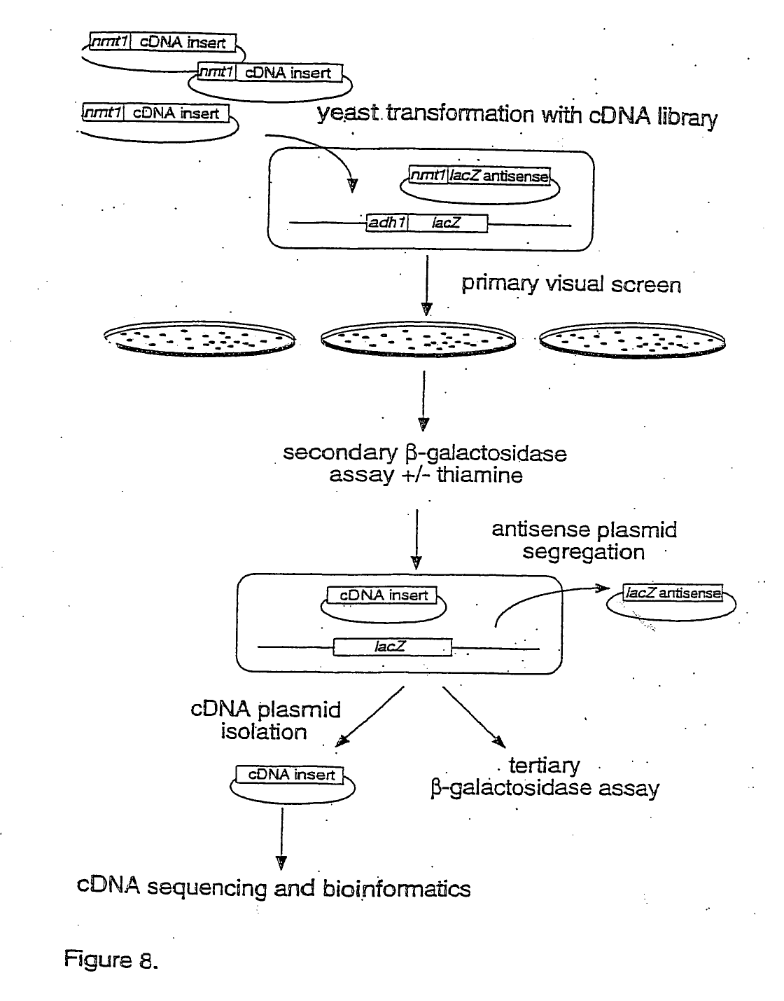

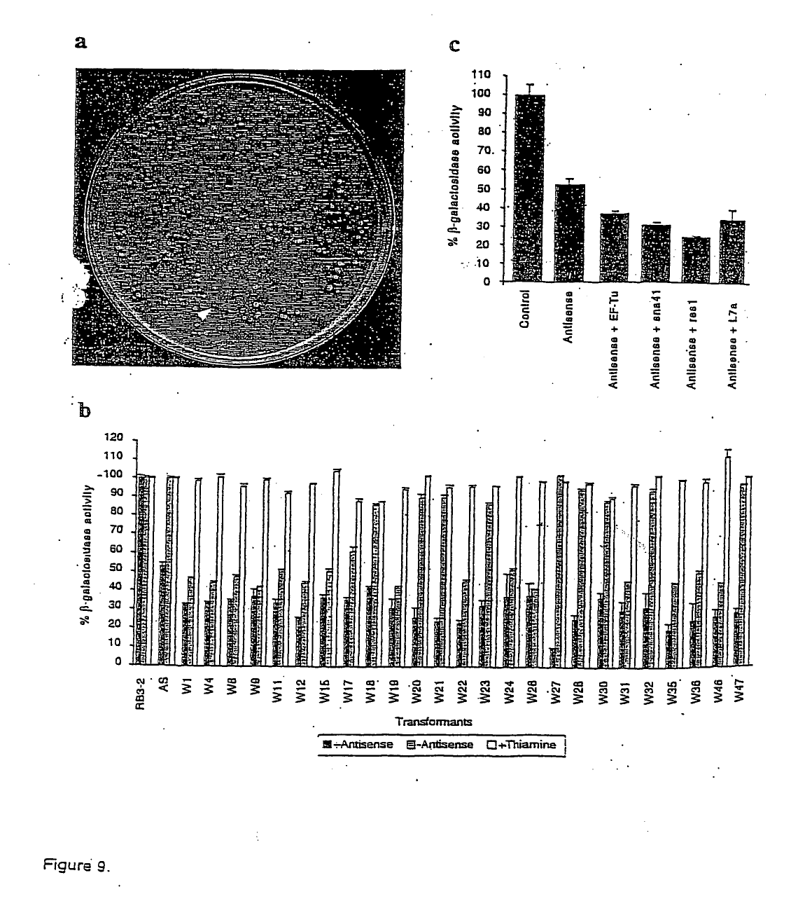

Over-expression of a fission yeast cDNA library (5) in an antisense lacZ expressing strain, revealed a series of transformants in which β-galactosidase activity was significantly reduced from that demonstrated in antisense RNA-expressing strains alone. The screening strategy is shown in FIG. 8. Transformants were grown on selective plates and overlayed with X-GAL-containing media to generate a blue phenotype which could be analysed visually in a semi-quantitative manner (FIG. 9A)(3). From 12,000 transformants screened, 48 were initially identified as having a reduced blue phenotype compared with background transformants. Of these, 25 demonstrated a reproducible enhancement in antisense RNA-mediated lacZ suppression when quantified by a liquid β-galactosidase assay (FIG. 9B). To demonstrate that the observed effects were due to transcription of the cDNA insert, transformants were grown in the presence of thiamine. This repression of the nmt1 promoter resulted in a return to control levels of β-galactosidase activity in all transformants (FIG. 9B). This indicated that the enhanced suppression was due to expression of the cDNA rather than other genetic events such as lacZ reporter mutations. To determine if the effect was dependent on the presence of dsRNA, the antisense plasmid was segregated out of the transformants and the strains were again assayed for β-galactosidase activity. Nine of the 25 transformants demonstrated a return to the level of β-galactosidase seen in the parental strain indicating that the effect was dsRNA-dependent (FIG. 9B). The cDNAs being expressed in these transformants were named RNAi enhancing sequences (res). All transformants displayed normal growth phenotypes indicating that over-expression of the resident cDNAs did not affect general biology of the cell. In the absence of antisense RNA the remaining 16 transformants retained a reduced β-galactosidase activity indicating that the enhanced gene silencing was not dependent on the presence of dsRNA. This suggests that the cDNA-encoded proteins in these strains could have been affecting lacZ at the level of transcription or the stability or function of the protein.

The library plasmids were recovered from the aes-containing strains (also referred to as res-containing strains) and their cDNA inserts sequenced. BLASTN and BLASTP analysis identified clones W18, W20, and W30 (named aes2) as homologues of domains 2 and 3 of the mitochondrial elongation factor Tu (EF Tu). EF Tu is an essential protein which plays a role in transporting tRNA to the A site in the ribosome for peptide elongation. The cDNA in transformants W21, W23, and W32 (named aes3) was homologous to a putative protein that was identified in a screen for fission yeast ORFs. Interestingly, the cDNA insert was also homologous to the antisense strand of the 3′ UTR of the fission yeast gene sna41 which has previously been shown to be involved in DNA replication. It is thus possible that aes3 may operate through more than one mechanism in enhancing antisense RNA activity. The cDNA in transformant W47 (named aes4) was homologous to the antisense strand of the ribosomal protein L7a, a component of the 60S ribosomal subunit. aes4 also contained a small ORF of unknown biological function. The inserts in transformants W27 and W28 (named aes1) shared homology with a putative protein from C. albicans that was identified in a screen for genes essential for cell growth. A tertiary quantitative β-galactosidase assay was performed to obtain accurate levels of gene silencing augmentation in the antisense lacZ strains co-expressing these unique factors (FIG. 9C). This analysis showed that these co-factors enhanced antisense suppression by up to 50%. These results demonstrated that over-expression of a cDNA library was an effective way of identifying novel co-factors that magnify the suppressive effect mediated by antisense RNA.

aes1 shared 43% identity with amino acids 4 to 202 of a C. albicans hypothetical protein (AJ390519). aes2 shared 99% identity with nucleotides 10452 to 9484 of the translation elongation factor EF Tu (AL049769). aes3 shared 94% identity with nucleotides 776 to 1145 of D89239 S. pombe ORF (D89239) and 93% identity with nucleotides 3246 to 2876 of the antisense strand of the DNA replication factor sna41 (AB001379). aes3 also contained a 220 nt stretch of a GA repeat sequence at its 3′ end. aes4 shared 99% identity with nucleotides 9678 to 8897 of the antisense strand of ribosomal protein L7a (AJ001133) and 99% identity with nucleoStides 1365 to 584 of the antisense strand of ribosomal protein L4 (AB005750). The reference numerals in brackets refer to accession numbers in the GeneBank database (the GeneBank database can be accessed from the following web site: http://www.ncbi.nlm.nih.gov/).

EF Tu is analogous to the eukaryotic EF1α and acts by transporting tRNA to the A site in the ribosome for peptide elongation. EF1α is an essential protein which has also been implicated in a large array of cellular activities including actin binding, microtubule severing, cellular transformation, cell senescence, protein ubiquitination, and protein folding. Detailed analysis of the EF Tu-expressing strain showed that it enhanced antisense RNA-mediated lacZ silencing by an additional 15% (FIG. 9C). It has been proposed that dsRNA is fragmented into 21-25 nt species by dsRNA-specific nucleases, amplified by RNA-dependent RNA polymerase, and dissociated by an ATP-dependent RNA helicase. The small antisense fragments are then free to attack homologous mRNA by RNA nuclease-mediated degradation. It has recently been suggested that an associative mechanism could catalyse the ATP-dependent exchange of the sense strand of the short dsRNA with the target mRNA. This may involve the transport of fragmented dsRNA to the ribosome where, in a competitive reaction with tRNA complexes, the complementary antisense strand binds to the mRNA. This would permit the association of the antisense strand of the dsRNA with the target mRNA when the latter is in a structurally exposed state and both inhibit translation and target the mRNA for ribonuclease degradation. Without wishing to be bound by any particular mechanism of action, EF Tu could act by binding to RNAi-dependent short dsRNA species and bring them to the site of action.

The protein encoded by sna41 has previously been shown to be involved in DNA replication. sna41 has low homology with CDC45 and might have DNA helicase properties which could facilitate the expression of complementary sequences. It is conceivable that the antisense plasmid and target DNA sequences may ectopically pair by intermolecular complementarity. Such pairing may inhibit RNA expression and consequently reduce the level of intracellular dsRNA. Similarly the intramolecular pairing of inverted repeat DNA sequences may also interfere with RNA expression. The over-expression of a protein with DNA helicase properties could facilitate the generation of more dsRNA which could in turn enhance the RNAi effect. Furthermore, CDC45 mutants show an increased rate of plasmid segregation. If plasmid loss is inhibited by over-expression of sna41 then more dsRNA may be generated leading to more effective RNAi in this system. In this light it is not unreasonable to expect that other proteins normally involved in DNA and/or RNA metabolism and function could also have a role in RNAi modulation and/or enhancement.

The L7a protein is part of the 60s ribosomal sub-unit. Without wishing to be bound by any particular mechanism of action, RNAi augmentation by over-expression of this protein is reasonable as it is hypothesised that the short dsRNA species may undergo strand displacement with target mRNA at the ribosome. The L7a ribosomal protein may act in RNAi by i) mediating docking of dsRNA or its unwound form into the A site of the ribosome, assisting in association of the antisense strand with the target mRNA, and/or shuttling of dsRNA to the ribosomal complex.

Nucleotide sequences representative of the aes factors referred to above are provided below and identified as Seq ID Nos 1 to 4:

DNA sequence for aes1 factor
(Seq ID No 1)
TTTAACTTAGTTCGCTTTGTTAAATTGGCCTCGAGGTCGACGTTAATTAA
GCCGCAATTGTAACAGTAGATTTTTTGCATCATTATTACTCTCCGAAACA
TGACTGAACACTCATTTAAGCAAATAGACGTGTTTTCTAATAAAGGTTTT
CGAGGTAATCCTGTTGCAGTTTTTTTTGATGCAGATAATTTATCACAAAA
GGAAATGCAGCAGATTGCCAAGTGGACAAATTTATCTGAGACAACATTTG
TTCAAAAGCCGACAATCGATAAAGCAGATTACAGACTTCGTATATTTACC
CCAGAATGTGAATTAAGCTTTGCTGGTCACCCAACAATTGGATCGTGCTT
TGCTGTTGTTGAAAGTGGATATTGTACTCCAAAAAACTGTAAAATTATTC
AGGAATGTTTAGCCGGTTTAGTTGAATTAACTATCGATGGGGAAAAGGAT
GAAGACACTTGGATTTCTTTCAAACTTCCGTATTACAAAATTTTACAGAC
TTCTGAAACTGCAATTTCAGAAGTAGAAAATGCATTGGGTATTCCTCTGA
ATTATAGTTCTCAAGTTTCTCCTCCTGTGTTAATAGATGATGGACCAAAG
TGGCTTGTAATTCAACTTCCAAACGCTACAGATGTGCTCAACCTCGTTCC
GAAATTTCAGTCCCTTTCCCAAGTTTGTAAAAACAATGATTGGATAGGCG
TCACCCGTCTTTGGTGAATTAGAAAAGACTCGTTTGAAAGCCCGAAGCTT
TGCGCCTTTAATACATGTCAATGAGGATCCGGCTTGCGGTAGTGGTGCAG
GAGCTGTCGGTGTGTATATTGGAAGCTCTCAAAAAACTCCAACTTCTCTA
TCATTTACGATTTCTCAAGGTACAAAATTAAGTAGACAAGCAATTTCCAA
AGTCAGCGTAGACGTTTCCTCCAATAAATCAATTGCTGTTTTTGTCGGTG
GACAGGCAAAAACTTGTATTTCTGGAAAATCGTTTATTTAATGTTTTTAT
TACAAATATTCACTTGCGAGTTTATTTTCCAATACTGAAGACTTTCAATC
AATAGCAAATATGCTACTCAAGGAAGTTCACTCATTCAAAAGCAATTGGT
TTACTATATCGTTTTTTCTAACTAGTTACTAGTCATTGAACAATCTACCG
AATGATAAAATGAAATTTTGGTTTTTCCCCGGGTAAAAGGAATGTCTCCC
TTGCCAGTACTGCTAGGGTTTTTCTTTCGAACTATAAGA
DNA sequence for aes2 factor
(Seq ID No 2)
AGTCCGCTTTGTTAAATTGGCCTCGAGGTCGACGTTAATTAAGCCTGATA
TGATCGAGCTTGTCGAAATGGAAATGCGTGAGCTACTCTCCGAATACGGA
TTTGATGGTGACAATACTCCAATTGTTAGCGGCAGTGCTTTATGTGCCTT
AGAGGGTCGTGAGCCTGAGATTGGTCTCAATAGTATTACTAAATTGATGG
AAGCTGTTGATAGTTATATTACTCTTCCTGAAAGAAAAACGGATGTCCCT
TTCTTGATGGCCATCGAGGACGTTTTTTCAATTTCAGGTCGCGGAACTGT
AGTCACTGGCCGTGTCGAGCGCGGTACTTTAAAGAAGGGTGCTGAAATCG
AAATCGTCGGTTATGGTAGCCATTTAAAGACTACCGTTACTGGAATTGAA
ATGTTCAAAAAGCAGCTTGATGCCGCCGTTGCCGGTGACAATTGTGGCCT
TTTACTTCGTTCTATCAAGCGAGAGCAATTAAAACGTGGAATGATTGTCG
CTCAACCAGGAACCGTTGCTCCTCATCAGAAATTCAAGGCATCATTCTAT
ATTTTGACAAAAGAGGAAGGAGGTCGTCGTACCCGGTTTCGTTGACAAGT
ATCGTCCCCAACTGTACAGTCCGTACTTCCGACGTTACTGTCGAACTTAC
CCACCCTGATCCTAACGACTCAACAAAATGGTTATGCCTGGAGACAATGT
CGAGATGATCTGTACGCTTATTCACCCCATTGTCATCGAAAAAGGACAAC
GCTTCACAGTTCGTGAGGGTGGAAGCACTGTAGGCACAGCTTTGGTTACT
GAACTTTTGGATTAGTGCATTTATGAACTTATTGGCTTTAAAAATTTTGC
ATGCTGAATACCAATATTATGTCCCTTCTCAGAATTCTATAACTACAGTG
TCATTATTGTAATAAGACTTTTGCATCCATTGACAATGGTATTTGATACT
TTTATAGTTTCTACTATTGTTAGCCAAAGTTATAAAACAAATAATAAAAT
AACGTTGAATCAAAAAAAAAAAAAAAAAAAGCGGCCGCGGATCCCCGGGT
AAAAGGAATGTCTCCCTTGCCAGTACTGCTAGGGTTTTTCTTTCAAACTA
TGGGA
DNA sequence for aes3 factor
(Seq ID No 3)
ATTTCAGACGCAATTCACATGGCTTTTGACTGTATTGCTATTCTTGTCGG
TTTAGTTGCTACGACGCTTGCCAAGATGCCTCTAAATTATGCTTACCCCT
TTGGATTTGCAAAAATTGAGGCTCTTTCGGGTTTCACTAATGGTATTTTT
TTAGTTTTGATTTCATTTTCTATCGTCGGCGAGGCATTATATAGGTTATT
TCATCCGCCCCAAATGAATACCGACCAATTGTTGTTGGTTAGTTTTTTGG
GCCTTGTTGTGAATTTGGTAGGTATCCTAGCGTTCAATCATGGGCATAAT
CATGATCATGGGTCTCATCACCATCATTCCCATAGTAATCATAGTATGTG
TCTGCCTAACACTACAAATGATATAAATATTTTTGAAGAGTTTGAAGAAG
AAAAAGATAATGTTGAAGCCCAGAAAATGGGCTATACGAATGACGATCAC
GTATCCCAACATGAACATACCCATGAGAATAGTCAGGAACATCACCATGA
GCATAACCACAATCATGATCACATCCATAAATACAATGAAAAATGCGACC
ATGAAAGCATAAGTCTCCAGAATTTAGACAATGATCATCACTGTCATCAT
CACCATGAAAATCATAATATGCATGGCATATTTCTGCATATTATCGCAGA
TACTATGGGCTCTGTTGGAGTTATTGTCTCTACTATATTAATACAGTGGT
TTTCATGGACCGGTTTTGATCCTTCGGCATCTCTAATAATTGCTGCATTA
ATATTTGTTTCTGTACTTCCATTAATTAAAGATTCGGCGAAGAATTTGCT
CTCTGTGACTGATCCAGAATCGGAATATTTATTGAAGCAGTGTTTGTCGA
ACATCAGTTTAAGTCACTCCGTTGTCAGTTTATCCAACCCTAAGTTCTGG
ACAAACGAAAGAGGTGAAGTGTATGGAATACTCCATATTCAGGTGAGCAT
AGACGGTGATTTAAACGTGGTTCGTAATGAAGTATTTAGGAAGCTCTCAA
TCGCTGTACCAAATTTAAAACACATTTGTATACAATCTGAACGGCCAAAC
AATTGCTGGTGTGGAAAATAGTTCTTACATCAGTTGATATCCATACTTAT
TTACGTGTAATTTTAATTAGATGAATTAATATTTTCTTTATTAGC
DNA sequence for aes4 factor
(Seq ID No 4)
TTTACTTTAGTCGCTTTGTTAAATTGGCCTCGAGGTCGACGTTAATTAAG
CTTTTTTTTTAAGAGATATAACATATGTCAACGCGTCATTGATTAACTAC
ATAACACGCCAATTATAAACTTCTCCCAAAAGAACTTAAGAATTTCCATT
TTCAATCCAGATGAATTTATTTAAGAGACGAACAGTAGCGGCAGCAGCCT
TAGCACGCTTAGCGAGCAAAGCTTGGGTCTTACCACCCATGATACCCACC
ACCCCACTTACGACGGGCTTTCGTCGTACTTAGCAGAGAAGTTAGCATCA
ACGGCGGAGACAATAGAAGCGAGTTCGTTCTTGTCTTCCTCACGGACCTC
AGTGACAGCTAAAACAGCAGCAGTCTTTTGGTGAATGACAGTACCAAGGC
GGGCCTTGTTCTTGACAATGGCATAAGGAACACCCATCTTCTTGCACAAA
GCAGGCAAGAAAACGACGAGTTCAATGGGGTCGACATCGCTGGCAATGAG
AACCAACTTAGCCTTCTTGGCCTCAATGAGAGCTACAACATGGTTCAAAC
CATATTTAACATTGTAAGGCTTCTTAGAGACGTCTTGAGCAGACTTGCCG
TTGGCAACAGCCTCGGCTTCAGCAACCAAACGTTGCTTCTTTTCAGCAGC
AGTCTCAGGACGGTACTTGTTAAGCAACTTGAAGACCTGAGTAGCAGTGT
TTTTGTCCAAAGTCTTCTGGAACTGAGCAATGGCAGGAGGAACCTTCAAA
CGCAAGTTCAAAATCTTGCGACGGCGTTGAAGGCGGATATACTCAGGCCA
CTTAACAAAACGGCTCAAGTCACGCTTAGGTTGGATGTCTTGTCCCCCGG
GTAAAAGGAATGTCTCCCTTGCCAGTACTGCTAGGGTTTTTCGTTCGAAT
AAGGCC

### Example 9

### Effect of Novel RNAi Factors on Antisense RNA and dsRNA-Mediated Gene Regulation

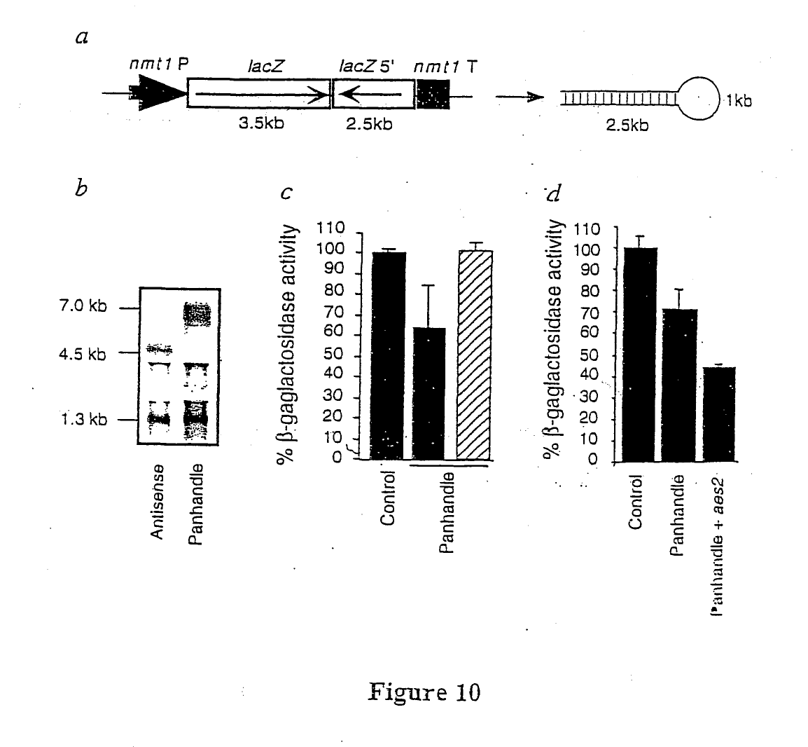

With recent studies on PTGS suggesting that antisense RNA, co-suppression, and dsRNA-mediated interference may share similar mechanisms, we wanted to determine whether over-expression of an aes factor would also enhance dsRNA-mediated regulation. Having demonstrated that dsRNA could mediate gene suppression in this fission yeast model, the effect of an antisense enhancing sequence on dsRNA-mediated regulation was tested. To this end, the aes2 gene was co-expressed with the lacZ panhandle construct in a yeast strain containing the lacZ target gene. Under these conditions, this transformant displayed an additional 30% suppression of β-galactosidase activity when compared to the transformant expressing only the panhandle lacZ RNA (see FIG. 10). This result shows that the aes2 gene, encoding the two domains of the mitochondrial elongation factor EF Tu, stimulated not only antisense RNA but also dsRNA-mediated gene inhibition.

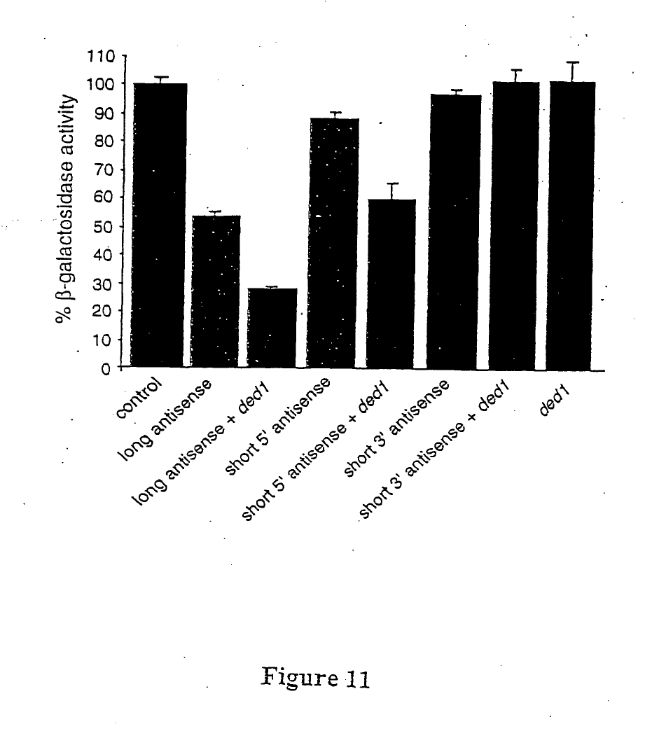

Further it was shown that these two forms of regulation are related by over-expressing the ATP-dependent RNA helicase ded1 in the presence of lacZ antisense RNA and showing that this helicase enhanced gene suppression by a further 50% compared to the control strain (example 6). ded1 was tested on both active and inactive antisense plasmids and demonstrated that ded1 augmentation of gene silencing was dependent on an active antisense RNA (see FIG. 11). This could be due to the absence of RNA duplex formation with the inactive antisense RNA and the consequent lack of a substrate for the RNA helicase.

The methods of the present invention have utility in demonstrating a range of RNAi efficacies, in identifying new factors which enhance or reduce gene silencing, in inhibiting gene expression or increasing sensitivity to antisense inhibition of gene expression, in the treatment or prevention of disorders which require inhibition or down-regulation of gene expression.

Although the present invention has been described with reference to specific examples and preferred embodiments it will be clear to those skilled in the art that variations and modifications which do not depart from the concept and the spirit of the invention described herein are also contemplated as being within the scope of the present invention.

### REFERENCES

1. Raponi, M., Atkins, D., Dawes, I. & Arndt, G. (2000) Antisense and Nucleic Acid Drug Development 10, 29-34.
2. Arndt, G., Atkins, D., Patrikakis, M. & Izant, J. (1995) Molecular and General Genetics 248, 293-300.
3. Arndt, G., Patrikakis, M. & Atkins, D. (2000) Nucleic Acids Research 28, e15.
4. Clarke, M., Patrikakis, M. & Atkins, D. (2000) Biochemical and Biophysical Research Communications 268, 8-13.
5. Moreno, S. & Nurse, P. (1994) Nature 367, 236-242.
6. Moreno, S., Klar, A. & Nurse, P. (1991) Methods in Enzymology 194, 795-823.
7. Maundrell, K. (1990) Journal of Biological Chemistry 265, 10857-10864.
8. Prentice, H. (1992) Nucleic Acids Research 20, 621.
9. Hoffman, C. & Winston, F. (1987) Gene 57, 267-272.
10. Rose, M., Winston, F. & Hieter, P. (1990) Methods in yeast genetics: A laboratory course manual (Cold Spring Harbor Laboratory Press, Cold Spring Harbor).
11. Maundrell, K. (1993) Gene 123, 127-1390.
12. Basi, G., Schmid, E. & Maundrell, K. (1993) Gene 123, 131-136.
13. Carr, A., MacNeill, S., Hayles, J. & Nurse, P. (1989) Molecular and General Genetics 218, 41-49.
14. Sambrook, J., Fritsch, E. & Maniatis, T. (1989) (Cold Spring Harbor Laboratory Press, Cold Spring Harbor, N.Y.).
15. Patrikakis, M., Izant, J. & Atkins, D. (1996) Current Genetics 30, 151-158.
16. Brun, C., Dubey, D. & Huberman, J. (1995) Gene 164, 173-177.
17. Heyer, W.-D., Sipiczki, M. & Kohli, J. (1986) Molecular and Cellular Biology 6, 80-89.

1. A method for inhibiting the expression of a target nucleic acid in a cell, which method comprises the steps of
(i) elevating in the cell the level of an RNAi factor, and
(ii) prior, concurrently with or subsequent to performing step (i), introducing into the cell a molecule which is, or gives rise to, an anti-sense nucleic acid directed toward at least a portion of the RNA transcript of the target nucleic acid, under conditions permitting the RNAi factor to increase the degree to which the anti-sense nucleic acid inhibits expression of the target nucleic acid.

2. A method of increasing cellular susceptibility to anti-sense-mediated inhibition of target nucleic acid expression, which method comprises elevating the level of an RNAi factor in a cell that expresses said target nucleic acid, with the proviso that the cell is to have prior, concurrently or subsequently introduced thereinto a molecule which is, or gives rise to, an anti-sense nucleic acid directed toward at least a portion of the RNA transcript of the target nucleic acid under conditions permitting the RNAi factor to increase the degree to which the anti-sense nucleic acid inhibits expression of the target nucleic acid.

3. A method for treating a subject suffering from a disorder whose alleviation is mediated by inhibiting the expression of a target nucleic acid, which method comprises the steps of
(i) elevating the level of an RNAi factor in the subject's cells where the target nucleic acid is expressed, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting the RNAi factor to increase the degree to which the anti-sense nucleic acid inhibits expression of the target nucleic acid, thereby treating the subject.

4. A method for inhibiting in a subject the onset of a disorder whose alleviation is mediated by inhibiting the expression of a target nucleic acid, which method comprises the steps of
(i) elevating the level of an RNAi factor in the subject's cells where the target nucleic acid would be expressed if the subject were suffering from the disorder, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting the RNAi factor to increase the degree to which the anti-sense nucleic acid would inhibit expression of the target nucleic acid were such expression to occur, thereby inhibiting in the subject the onset of the disorder.

5. A method of determining whether inhibiting the expression of a particular target nucleic acid or the activity of its product may alleviate a disorder, which method comprises the steps of
(i) elevating the level of an RNAi factor in a cell whose phenotype correlates with that of a cell from a subject having the disorder;
(ii) prior, concurrently with or subsequent to performing step (i), introducing into the cell a molecule which is, or gives rise to, an anti-sense nucleic acid directed toward at least a portion of the RNA transcript of the target nucleic acid under conditions permitting the RNAi factor to increase the degree to which the anti-sense nucleic acid inhibits expression of the target nucleic acid; and
(iii) determining whether the cell's phenotype now correlates with that of a cell from a subject in whom the disorder has been alleviated or the disorder is not evident, thereby determining whether inhibiting the expression of the target nucleic acid or the activity of its product may alleviate the disorder.

6. A method according to any one of claims 1 to 5, wherein the target nucleic acid is an exogenous nucleic acid or a part thereof.

7. A method according to any one of claims 1 to 6, wherein the level of the RNAi factor is elevated by introducing into the cell additional copies of, or agents which give rise to, the RNAi factor.

8. A method according to any one of claims 1 to 7, wherein the factor is selected from the group consisting of a gene, cDNA, RNA or a protein.

9. A method according to any one of claims 1 to 8, wherein the factor is selected from the group consisting of a transcriptional activator of the antisense nucleic acid, a component of the RNAi machinery, a component of the DNA replication machinery and a component of translational machinery.

10. A method according to any one of claims 1 to 9, wherein the RNAi factor is an res sequence.

11. A method according to claim 10, wherein the factor is selected from the group consisting of ATP-dependent RNA helicase (ded1), transcriptional factor thi1, DNA replication protein sna41, ribosomal protein L7a, elongation factor EF-Tu and res1 as herein defined.

12. A method according to claim 11, wherein the res sequence is obtainable from transformed cells designated herein W18, W20, W21, W23, W27, W28, W30, W32 and W47.

13. A method according to claim 11, wherein the res sequence is represented by Seq ID Nos 1 to 4.

14. A method according to any one of claims 1 to 13, wherein the cell is a eukaryotic cell.

15. A method according to 14, wherein the eukaryotic cell is a mammalian.

16. A method according to claim 1 or claim 2, wherein the cell is a Schizosaccharomyces pombe cell.

17. A method according to any one of claims 1 to 16, wherein the antisense nucleic acid corresponds to a part of the target nucleic acid.

18. A pharmaceutical composition for use in performing the method of any one of claims 2 to 17 comprising
(i) an expressible nucleic acid encoding, or capable of increasing or decreasing the expression of, an RNAi factor;
(ii) a nucleic acid encoding a molecule which is, or gives rise to, an anti-sense nucleic acid directed toward at least a portion of the RNA transcript of the target nucleic acid; and
(iii) a pharmaceutically acceptable carrier,
wherein the nucleic acids of (i) and (ii) may be situated on the same or different molecules.

19. A pharmaceutical composition for use in performing the method of any one of claims 2 to 17 comprising
(i) an nucleic acid which is the target nucleic acid or a part thereof, or an expressible nucleic acid encoding a factor capable of elevating the intracellular level of the target nucleic acid;
(ii) a nucleic acid encoding a molecule which is, or gives rise to, an anti-sense nucleic acid directed toward at least a portion of the RNA transcript of the target nucleic acid; and
(iii) a pharmaceutically acceptable carrier,
wherein the nucleic acids of (i) and (ii) may be situated on the same or different molecules.

20. A cell having increased susceptibility to anti-sense-mediated inhibition of a target nucleic acid expression, which cell (i) expresses a target nucleic acid and (ii) comprises an elevated level of an RNAi factor, with the proviso that the cell is to have introduced thereinto a molecule which is, or gives rise to, an anti-sense nucleic acid directed toward at least a portion of the RNA transcript of the target nucleic acid under conditions permitting the RNAi factor to increase the degree to which the anti-sense nucleic acid inhibits expression of the target nucleic acid.

21. A cell according to claim 20, wherein the cell is a eukaryotic cell.

22. A cell according to claim 20 or claim 21, wherein the cell is a Schizosaccharomyces pombe cell.

23. A method for inhibiting the expression of a target nucleic acid in a cell, which method comprises the steps of
(i) augmenting the level of the target nucleic acid or a part thereof in the cell, and
(ii) prior, concurrently with or subsequent to performing step (i), introducing into the cell a molecule which is, or gives rise to, an anti-sense nucleic acid directed toward at least a portion of the RNA transcript of said target nucleic acid, under conditions permitting an increase in the degree to which the anti-sense nucleic acid inhibits expression of said target nucleic acid.

24. A method of increasing cellular susceptibility to anti-sense-mediated inhibition of a target nucleic acid expression, which method comprises augmenting the level of the target nucleic acid or a part thereof in a cell expressing the target nucleic acid, with the proviso that the cell is to have prior, concurrently or subsequently introduced thereinto a molecule which is, or gives rise to, an anti-sense nucleic acid directed toward at least a portion of the RNA transcript of the target nucleic acid under conditions permitting the increase in the degree to which the anti-sense nucleic acid inhibits expression of the target nucleic acid.

25. A method for treating a subject suffering from a disorder whose alleviation is mediated by inhibiting the expression of a target nucleic acid, which method comprises the steps of
(i) augmenting the level of said target nucleic acid or a part thereof in the subject's cells where the target nucleic acid is expressed, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting an increase in the degree to which the anti-sense nucleic acid inhibits expression of the target nucleic acid, thereby treating the subject.

26. A method for treating a subject suffering from a disorder whose alleviation is mediated by inhibiting the expression of a target nucleic acid, which method comprises the steps of
(i) augmenting the level of the target nucleic acid or a part thereof in the subject's cells where the target nucleic acid is expressed, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting an increase in the degree to which the anti-sense nucleic acid inhibits expression of the target nucleic acid, thereby treating the subject.

27. A method for inhibiting in a subject the onset of a disorder whose alleviation is mediated by inhibiting the expression of a target nucleic acid, which method comprises the steps of
(i) augmenting the level of the target nucleic acid or a part thereof in the subject's cells where the target nucleic acid would be expressed if the subject were suffering from the disorder, and
(ii) prior, concurrently with or subsequent to performing step (i), introducing into such cells a molecule which is, or gives rise to, an anti-sense nucleic acid directed toward at least a portion of the RNA transcript of the target nucleic acid, under conditions permitting an increase in the degree to which the anti-sense nucleic acid would inhibit expression of the target nucleic acid were such expression to occur, thereby inhibiting in the subject the onset of the disorder.

28. A method of determining whether inhibiting the expression of a particular target nucleic acid or the activity of its product may alleviate a disorder, which method comprises the steps of
(i) augmenting the level of the target nucleic acid in a cell whose phenotype correlates with that of a cell from a subject having the disorder;
(ii) prior, concurrently with or subsequent to performing step (i), introducing into the cell a molecule which is, or gives rise to, an anti-sense nucleic acid directed toward at least a portion of the RNA transcript of the target nucleic acid under conditions permitting an increase in the degree to which the anti-sense nucleic acid inhibits expression of the target nucleic acid; and
(iii) determining whether the cell's phenotype now correlates with that of a cell from a subject in whom the disorder has been alleviated or the disorder is not evident, thereby determining whether inhibiting the expression of the target nucleic acid or the activity of its product may alleviate the disorder.

29. A method according to any one of claims 23 to 28, wherein the target nucleic acid is an exogenous nucleic acid or a part thereof.

30. A method according to any one of claims 23 to 29, wherein the level of the target nucleic acid is augmented by introducing into the cell additional copies of, or agents which are capable of inducing intracellular over-expression of, the target nucleic acid.

31. A method according to any one of claims 23 to 30, wherein the cell is a eukaryotic cell.

32. A method according to 31, wherein the eukaryotic cell is a mammalian.

33. A method according to claim 23 or claim 24, wherein the cell is a Schizosaccharomyces pombe cell.

34. A method according to any one of claims 23 to 33, wherein the antisense nucleic acid corresponds to a part of the target nucleic acid.

35. A cell having increased susceptibility to anti-sense-mediated inhibition of a target nucleic acid expression, which cell (i) expresses said target nucleic acid and (ii) comprises an elevated level of said target nucleic acid, with the proviso that the cell is to have introduced thereinto a molecule which is, or gives rise to, an anti-sense nucleic acid directed toward at least a portion of the RNA transcript of the target nucleic acid under conditions permitting the RNAi factor to increase the degree to which the anti-sense nucleic acid inhibits expression of the target nucleic acid.

36. A cell according to claim 35, wherein the cell is a eukaryotic cell.

37. A cell according to claim 35 or claim 36, wherein the cell is a Schizosaccharomyces pombe cell.

38. Method of identifying a cellular factor capable of effecting and/or modulating expression of a target nucleic acid in a cell having the target nucleic acid and a nucleic acid which is an antisense of the target nucleic acid or part thereof, which method comprises over-expressing said factor in the cell and wherein the expression of the target nucleic acid is capable of being enhanced or only partially suppressed.

39. A factor identified by the method of claim 38.

40. A factor according to claim 39, wherein the factor is selected from the group consisting of a gene, cDNA, RNA or a protein.

41. A factor according to claim 39 or claim 40, wherein the factor is selected from the group consisting of a transcriptional activator or the antisense nucleic acid, a component of the RNAi machinery, a component of the DNA replication machinery and a component of translational machinery.

42. A factor according to any one of claims 39 to 41, wherein the factor is an res sequence.

43. A factor according to claim 42, wherein the factor is selected from the group consisting of ATP-dependent RNA helicase (ded1), transcriptional factor thi1, DNA replication protein sna41, ribosomal protein L7a, elongation factor EF-Tu and res1 as herein defined.

44. An RNAi factor which is an res sequence obtainable from transformed cells designated herein W18, W20, W21, W23, W27, W28, W30, W32 and W47.

45. An RNAi factor which is an res sequence represented by Seq ID Nos 1 to 4.

46. A Schizosaccharomyces pombe cell having a target nucleic acid or a part thereof and a antisense nucleic acid or a part thereof which corresponds to the target nucleic acid or a part thereof, wherein the expression of the target nucleic acid is capable of being enhanced or only partially suppressed.


SEQUENCE LISTING (11 sequences)

SEQ ID NO: 1 - DNA, 1239 bp, fission yeast
tttaacttagttcgctttgttaaattggcctcgaggtcgacgttaattaagccgcaattgtaacagtagattttttgcatcattattactctccgaaacatgactgaacactcatttaagcaaatagacgtgttttctaataaaggttttcgaggtaatcctgttgcagttttttttgatgcagataatttatcacaaaaggaaatgcagcagattgccaagtggacaaatttatctgagacaacatttgttcaaaagccgacaatcgataaagcagattacagacttcgtatatttaccccagaatgtgaattaagctttgctggtcacccaacaattggatcgtgctttgctgttgttgaaagtggatattgtactccaaaaaactgtaaaattattcaggaatgtttagccggtttagttgaattaactatcgatggggaaaaggatgaagacacttggatttctttcaaacttccgtattacaaaattttacagacttctgaaactgcaatttcagaagtagaaaatgcattgggtattcctctgaattatagttctcaagtttctcctcctgtgttaatagatgatggaccaaagtggcttgtaattcaacttccaaacgctacagatgtgctcaacctcgttccgaaatttcagtccctttcccaagtttgtaaaaacaatgattggataggcgtcacccgtctttggtgaattagaaaagactcgtttgaaagcccgaagctttgcgcctttaatacatgtcaatgaggatccggcttgcggtagtggtgcaggagctgtcggtgtgtatattggaagctctcaaaaaactccaacttctctatcatttacgatttctcaaggtacaaaattaagtagacaagcaatttccaaagtcagcgtagacgtttcctccaataaatcaattgctgtttttgtcggtggacaggcaaaaacttgtatttctggaaaatcgtttatttaatgtttttattacaaatattcacttgcgagtttattttccaatactgaagactttcaatcaatagcaaatatgctactcaaggaagttcactcattcaaaagcaattggtttactatatcgttttttctaactagttactagtcattgaacaatctaccgaatgataaaatgaaattttggtttttccccgggtaaaaggaatgtctcccttgccagtactgctagggtttttctttcgaactataaga

SEQ ID NO: 2 - DNA, 1105 bp, fission yeast
agtccgctttgttaaattggcctcgaggtcgacgttaattaagcctgatatgatcgagcttgtcgaaatggaaatgcgtgagctactctccgaatacggatttgatggtgacaatactccaattgttagcggcagtgctttatgtgccttagagggtcgtgagcctgagattggtctcaatagtattactaaattgatggaagctgttgatagttatattactcttcctgaaagaaaaacggatgtccctttcttgatggccatcgaggacgttttttcaatttcaggtcgcggaactgtagtcactggccgtgtcgagcgcggtactttaaagaagggtgctgaaatcgaaatcgtcggttatggtagccatttaaagactaccgttactggaattgaaatgttcaaaaagcagcttgatgccgccgttgccggtgacaattgtggccttttacttcgttctatcaagcgagagcaattaaaacgtggaatgattgtcgctcaaccaggaaccgttgctcctcatcagaaattcaaggcatcattctatattttgacaaaagaggaaggaggtcgtcgtacccggtttcgttgacaagtatcgtccccaactgtacagtccgtacttccgacgttactgtcgaacttacccaccctgatcctaacgactcaacaaaatggttatgcctggagacaatgtcgagatgatctgtacgcttattcaccccattgtcatcgaaaaaggacaacgcttcacagttcgtgagggtggaagcactgtaggcacagctttggttactgaacttttggattagtgcatttatgaacttattggctttaaaaattttgcatgctgaataccaatattatgtcccttctcagaattctataactacagtgtcattattgtaataagacttttgcatccattgacaatggtatttgatacttttatagtttctactattgttagccaaagttataaaacaaataataaaataacgttgaatcaaaaaaaaaaaaaaaaaaagcggccgcggatccccgggtaaaaggaatgtctcccttgccagtactgctagggtttttctttcaaactatggga

SEQ ID NO: 3 - DNA, 1145 bp, fission yeast
atttcagacgcaattcacatggcttttgactgtattgctattcttgtcggtttagttgctacgacgcttgccaagatgcctctaaattatgcttacccctttggatttgcaaaaattgaggctctttcgggtttcactaatggtatttttttagttttgatttcattttctatcgtcggcgaggcattatataggttatttcatccgccccaaatgaataccgaccaattgttgttggttagttttttgggccttgttgtgaatttggtaggtatcctagcgttcaatcatgggcataatcatgatcatgggtctcatcaccatcattcccatagtaatcatagtatgtgtctgcctaacactacaaatgatataaatatttttgaagagtttgaagaagaaaaagataatgttgaagcccagaaaatgggctatacgaatgacgatcacgtatcccaacatgaacatacccatgagaatagtcaggaacatcaccatgagcataaccacaatcatgatcacatccataaatacaatgaaaaatgcgaccatgaaagcataagtctccagaatttagacaatgatcatcactgtcatcatcaccatgaaaatcataatatgcatggcatatttctgcatattatcgcagatactatgggctctgttggagttattgtctctactatattaatacagtggttttcatggaccggttttgatccttcggcatctctaataattgctgcattaatatttgtttctgtacttccattaattaaagattcggcgaagaatttgctctctgtgactgatccagaatcggaatatttattgaagcagtgtttgtcgaacatcagtttaagtcactccgttgtcagtttatccaaccctaagttctggacaaacgaaagaggtgaagtgtatggaatactccatattcaggtgagcatagacggtgatttaaacgtggttcgtaatgaagtatttaggaagctctcaatcgctgtaccaaatttaaaacacatttgtatacaatctgaacggccaaacaattgctggtgtggaaaatagttcttacatcagttgatatccatacttatttacgtgtaattttaattagatgaattaatattttctttattagc

SEQ ID NO: 4 - DNA, 906 bp, fission yeast
tttactttagtcgctttgttaaattggcctcgaggtcgacgttaattaagctttttttttaagagatataacatatgtcaacgcgtcattgattaactacataacacgccaattataaacttctcccaaaagaacttaagaatttccattttcaatccagatgaatttatttaagagacgaacagtagcggcagcagccttagcacgcttagcgagcaaagcttgggtcttaccacccatgatacccaccaccccacttacgacgggctttcgtcgtacttagcagagaagttagcatcaacggcggagacaatagaagcgagttcgttcttgtcttcctcacggacctcagtgacagctaaaacagcagcagtcttttggtgaatgacagtaccaaggcgggccttgttcttgacaatggcataaggaacacccatcttcttgcacaaagcaggcaagaaaacgacgagttcaatggggtcgacatcgctggcaatgagaaccaacttagccttcttggcctcaatgagagctacaacatggttcaaaccatatttaacattgtaaggcttcttagagacgtcttgagcagacttgccgttggcaacagcctcggcttcagcaaccaaacgttgcttcttttcagcagcagtctcaggacggtacttgttaagcaacttgaagacctgagtagcagtgtttttgtccaaagtcttctggaactgagcaatggcaggaggaaccttcaaacgcaagttcaaaatcttgcgacggcgttgaaggcggatatactcaggccacttaacaaaacggctcaagtcacgcttaggttggatgtcttgtcccccgggtaaaaggaatgtctcccttgccagtactgctagggtttttcgttcgaataaggcc

SEQ ID NO: 5 - DNA, 12 bp, Artificial Sequence
  Oligonucleotide primer
ccgggcggccgc

SEQ ID NO: 6 - DNA, 30 bp, Artificial Sequence
  Oligonucleotide primer
atgcggccgcaattcccggggatcgaaaga

SEQ ID NO: 7 - DNA, 29 bp, Artificial Sequence
  Oligonucleotide primer
atgcggccgcaatggggtcgcttcactta

SEQ ID NO: 8 - DNA, 30 bp, Artificial Sequence
  Oligonucleotide primer
atgggatcccaaccaaacacttcaactcag

SEQ ID NO: 9 - DNA, 30 bp, Artificial Sequence
  Oligonucleotide primer
atgggatcctcagaagcctgtgcataacac

SEQ ID NO: 10 - DNA, 30 bp, Artificial Sequence
  Oligonucleotide primer
atgagatctgtggttggtattctagagaga

SEQ ID NO: 11 - DNA, 30 bp, Artificial Sequence
  Oligonucleotide primer
atgagatctaacaaagacctgcaaaaaacc